(19) United States
(12) Patent Application Publication
(10) Pub. No.: US 2024/0285407 A1
(43) Pub. Date: Aug. 29, 2024

(54) VALVE STENT FOR INTRAVASCULAR REPERFUSION THERAPY

(71) Applicant: KONINKLIJKE PHILIPS N.V., EINDHOVEN (NL)

(72) Inventors: James David CEZO, COLORADO SPRINGS, CO (US); Arjen VAN DER HORST, TILBURG (NL)

(21) Appl. No.: 18/693,487

(22) PCT Filed: Sep. 19, 2022
(86) PCT No.: PCT/EP2022/075883

§ 371 Date: Mar. 19, 2024

Related U.S. Application Data
(60) Provisional application No. 63/246,904, filed on Sep. 22, 2021.

(51) Int. Cl. A61F 2/24; A61B 8/04; A61B 8/06; A61B 8/12; A61F 2/48; A61F 2/95

(52) U.S. Cl. CPC A61F 2/2476; A61B 8/04; A61B 8/06; A61B 8/12; A61F 2/2418; A61F 2/484; A61F 2002/9528; A61F 2250/001

(57) ABSTRACT

A system includes a stent positionable within a coronary vein. The stent includes a valve and a stent body coupled to the valve and arranged to transition between a collapsed state and an expanded state. The system includes an intravascular reperfusion therapy device with a sensor and a catheter with a lumen shaped to receive the stent. The intravascular reperfusion therapy device delivers reperfusion therapy to a myocardium of a heart. The system includes a processor circuit that receives physiological data from the sensor, determines a progression of the reperfusion therapy, controls a configuration of the valve and/or the expansion state of the stent such that back pressure is controlled. With the stent in the expanded state, the stent body contacts a wall of the coronary vein and the valve obstructs blood flow in a first direction to generate the back pressure in an opposite, second direction.

### TECHNICAL FIELD

The present disclosure relates generally to a stent with a valve (e.g., a valve stent) arranged to intravascularly deliver a reperfusion therapy to an area of a heart of a patient. In particular, the disclosure relates to a stent arranged, to deliver the reperfusion therapy to the area, to obstruct a blood vessel of a patient in a first direction to generate a back pressure in an opposite, second direction in the blood vessel.

### BACKGROUND

A percutaneous coronary intervention (PCI) may be utilized to treat a blockage (e.g., an occlusion, a lesion, a stenosis, and/or the like) within a blood vessel. The PCI may include a therapeutic procedure, such as administration of a drug, angioplasty, placement of a stent, and/or the like, that reduces a size of the blockage or opens (e.g., widens) the lumen of the affected blood vessel. To that end, PCI can restore blood flow through a blood vessel and to tissue that receives blood/oxygen via the blood vessel. Moreover, before a PCI therapy is delivered, the reduction in blood flow caused by a blockage within a blood vessel may cause tissue that receives blood/oxygen from the vessel to experience ischemia. Accordingly, PCI may restore or increase blood flow to tissue that experienced ischemia, which may restore the health of the tissue. However, even after a PCI therapy is delivered, blood/oxygen may not always suitably re-perfuse through tissue that has experienced ischemia. In particular, the increase and/or reintroduction of blood flow through the ischemic tissue may trigger an inflammatory response and/or oxidative damage, known as reperfusion injury, along with or in place of restoration of normal function of the tissue.

### SUMMARY

Disclosed herein is a stent configured to be removably positioned (e.g., implanted) within a patient's body and to intravascularly deliver a reperfusion therapy targeting an area of the patient's body, such as a portion of the myocardium of the heart of the patient. The stent may include a valve coupled to a body of the stent and arranged to obstruct a flow of blood in a first direction in a blood vessel of the patient to generate a back pressure in an opposite, second direction in the blood vessel, thereby delivering the reperfusion therapy. The valve may obstruct the flow of blood by restricting an area (e.g., a diameter) available flow the blood to flow through within the blood vessel. Further, the stent may be deployed (e.g., positioned) within the blood vessel by an intravascular reperfusion therapy device, which may deploy the stent from within a lumen of a catheter, for example. Moreover, the stent, the intravascular reperfusion therapy device, and a processing system (e.g., processing circuit) may be included in a system configured to evaluate (e.g., assess) and control (e.g., modify) a progress of the reperfusion therapy. The processing system may receive physiological data (e.g., flow data, pressure data, and/or the like) representative of blood flow through the blood vessel from a sensor positioned intravascularly (e.g., on the intravascular reperfusion therapy device and/or the stent). The processing system may determine a progression of the reperfusion therapy delivered to the targeted area based on the physiological data, which may indicate an effect of the reperfusion therapy on the area. Moreover, based on the progression of the reperfusion therapy, the processing system may control (e.g., modify or maintain) the reperfusion therapy delivered by the intravascular device. In particular, the processing system may control expansion (e.g., deployment from the intravascular reperfusion therapy device) of the stent and/or a configuration of the valve of the stent such that obstruction of the blood vessel, the generated back pressure, and delivery of the reperfusion therapy is controlled. In this way, the processing system may use a feedback loop with the sensor to adaptively monitor and control delivery of the reperfusion therapy provided by the stent. In cases where the intravascular reperfusion therapy device is positioned within a coronary vein, for example, when a determined progression of the reperfusion therapy indicates that blood flow to the targeted area is improving and/or that the reperfusion therapy has achieved a particular outcome, the processing system may control the valve to gradually transition from a configuration having a first diameter (e.g., causing a first degree of obstruction) to a different, second configuration having a second, larger diameter (e.g., causing a second, lesser degree of obstruction) to decrease the venous obstruction or complete (e.g., terminate) the administration of the reperfusion therapy.

In an exemplary aspect, a system is provided. The system includes a stent configured to be positioned within a coronary vein of a patient, comprising: a valve; and a stent body coupled to the valve and the retrieval hook and arranged to transition an expansion state of the stent between a collapsed state and an expanded state; an intravascular reperfusion therapy device configured to be positioned within the coronary vein, wherein the intravascular reperfusion therapy device comprises a sensor and a catheter comprising a lumen shaped to receive the stent, wherein the intravascular reperfusion therapy device is configured to deliver, using the stent, reperfusion therapy to a myocardium of a heart of the patient associated with the coronary vein; and a processor circuit in communication with the intravascular reperfusion therapy device and configured to: receive, from the sensor, physiological data associated with blood flow through the coronary vein; determine, based on the physiological data, a progression of the reperfusion therapy delivered to the myocardium of the heart; and control, based on the progression of the reperfusion therapy, at least one of a configuration of the valve or the expansion state of the stent such that back pressure within the coronary vein is controlled, wherein, with the stent in the expanded state: the stent body is configured to contact a wall of the coronary vein; and the valve is arranged to deliver the reperfusion therapy by obstructing blood flow in a first direction in the coronary vein to generate the back pressure in an opposite, second direction in the coronary vein.

In some aspects, with the stent in the expanded state, the valve transitions a diameter of the stent from a first diameter to a second diameter less than the first diameter. In some aspects, the valve comprises a windsock valve. In some aspects, the valve comprises a duckbill valve. In some aspects, the valve is coupled to a proximal end of the stent body. In some aspects, the stent further comprises a retrieval hook such that the stent is configured to be removably positioned within the coronary vein, and the retrieval hook is coupled to a proximal end of the stent body. In some aspects, the retrieval hook is arranged such that, responsive to a mechanical force on the retrieval hook with the stent in the expanded state, the stent body transitions the stent to the collapsed state. In some aspects, in a first configuration, the valve is arranged to obstruct the blood flow in the first direction to a first degree, and, in a different, second configuration, the valve is arranged to obstruct the blood flow in the first direction to a different, second degree. In some aspects, the valve comprises an electroactive polymer configured to transition the valve from the first configuration to the second configuration in response to an electrical signal, and to control the configuration of the valve, the processor circuit is configured to control the electrical signal. In some aspects, the processor circuit is further configured to: output, to display in communication with the processor circuit, a visual representation of the progression of the reperfusion therapy. In some aspects, the sensor comprises a flow sensor, wherein the physiological data comprises a blood flow rate. In some aspects, the sensor comprises a pressure sensor, and the physiological data comprises pressure data. In some aspects, the sensor comprises an ultrasound transducer positioned at a distal portion of the catheter. In some aspects, the stent body comprises at least one of a wire mesh or a self-expanding material. In some aspects, the coronary vein comprises a coronary sinus. In some aspects, to determine the progression of the reperfusion therapy, the processor circuit is configured to: determine a derivative of the physiological data with respect to time.

In an exemplary aspect, a system is provided. The system includes a stent configured to be positioned within a coronary vein of a patient, comprising: a valve; and a stent body coupled to the valve and arranged to transition an expansion state of the stent between a collapsed state and an expanded state; an intravascular reperfusion therapy device configured to be positioned within the coronary vein, wherein the intravascular reperfusion therapy device comprises: a flow sensor positioned at a distal portion of the intravascular reperfusion therapy device; and a catheter comprising a lumen shaped to receive the stent, wherein the intravascular reperfusion therapy device is configured to deliver, using the stent, reperfusion therapy to a myocardium of a heart of the patient associated with the coronary vein; and a processor circuit in communication with the intravascular reperfusion therapy device and configured to: receive, from the flow sensor, flow data associated with blood flow through the coronary vein; determine, based on the flow data, a progression of the reperfusion therapy delivered to the myocardium of the heart; and control, based on the progression of the reperfusion therapy, at least one of a configuration of the valve or the expansion state of the stent such that back pressure within the coronary vein is controlled, wherein, with the stent in the expanded state: the stent body is configured to contact a wall of the coronary vein; and the valve is arranged to deliver the reperfusion therapy by obstructing blood flow in a first direction in the coronary vein to generate the back pressure in an opposite, second direction in the coronary vein, wherein the valve is arranged to obstruct the blood flow in the first direction to a first degree in a first configuration of the configuration of the valve and the valve is arranged to obstruct the blood flow in the first direction to a different, second degree in a second configuration of the configuration of the valve.

In an exemplary aspect, a stent removably positionable within a coronary vein is provided. The stent includes a valve; a retrieval hook; and a self-expanding material coupled to the valve and the retrieval hook and arranged to transition the stent between a collapsed state and an expanded state, wherein, with the stent in the expanded state: the self-expanding material is configured to contact a wall of the coronary vein; and the valve is arranged to obstruct blood flow in a first direction in the coronary vein to generate back pressure in an opposite, second direction in the coronary vein.

In an exemplary aspect, an apparatus is provided. The apparatus includes a stent configured to be positioned within a coronary vein of a patient, comprising: a valve; and a stent body coupled to the valve and arranged to transition an expansion state of the stent between a collapsed state and an expanded state; an intravascular reperfusion therapy device configured to be positioned within the coronary vein, wherein the intravascular reperfusion therapy device comprises a catheter comprising a lumen shaped to receive the stent, wherein the intravascular reperfusion therapy device is configured to deliver, using the stent, reperfusion therapy to a myocardium of a heart of the patient associated with the coronary vein, wherein, with the stent in the expanded state: the stent body is configured to contact a wall of the coronary vein; and the valve is arranged to deliver the reperfusion therapy by obstructing blood flow in a first direction in the coronary vein to generate the back pressure in an opposite, second direction in the coronary vein.

In some aspects, in a first configuration, the valve is arranged to obstruct the blood flow in the first direction to a first degree, and, in a different, second configuration, the valve is arranged to obstruct the blood flow in the first direction to a different, second degree. In some aspects, the valve comprises an electroactive polymer configured to transition the valve from the first configuration to the second configuration in response to an electrical signal. In some aspects, the system further comprises a processor circuit configured to provide the electrical signal to control the configuration of the valve.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic view of a system, in accordance with at least one embodiment of the present disclosure.

FIG. 2 is a schematic diagram of a processor circuit, in accordance with at least one embodiment of the present disclosure.

FIG. 3A is diagram of a human heart with an obstruction, in accordance with at least one embodiment of the present disclosure.

FIG. 3B is diagram of the human heart following a percutaneous coronary intervention (PCI), in accordance with at least one embodiment of the present disclosure.

FIG. 3C is diagram of the human heart following a reperfusion therapy, in accordance with at least one embodiment of the present disclosure.

FIG. 4 is a schematic diagram of a portion of a heart, in accordance with at least one embodiment of the present disclosure.

FIG. 5 is a schematic diagram of a portion of a heart, in accordance with at least one embodiment of the present disclosure.

FIG. 6A diagrammatic cross-sectional side view of an intravascular reperfusion therapy device with a stent in a first expansion state, in accordance with at least one embodiment of the present disclosure.

FIG. 6B is a diagrammatic side view of the intravascular reperfusion therapy device with the stent in a second expansion state, in accordance with at least one embodiment of the present disclosure.

FIG. 7A is a diagrammatic side view of a stent with a valve, in accordance with at least one embodiment of the present disclosure.

FIG. 7B is a diagrammatic side view of an intravascular reperfusion therapy device including a stent with a valve, in accordance with at least one embodiment of the present disclosure.

FIG. 8 is a flow diagram of a method for evaluating a progression of a reperfusion therapy, in accordance with at least one embodiment of the present disclosure.

FIG. 9 is a schematic diagram of a portion of a heart, in accordance with at least one embodiment of the present disclosure.

FIG. 10 is a diagrammatic, schematic view of a screen display, in accordance with at least one embodiment of the present disclosure.

FIG. 11 is a schematic diagram of a portion of a heart, in accordance with at least one embodiment of the present disclosure.

FIG. 12A is a front view of an intravascular reperfusion therapy device, in accordance with at least one embodiment of the present disclosure.

FIG. 12B is a front view of an intravascular reperfusion therapy device, in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

Aspects of the present disclosure can include features described in App. No. 63/246,946, filed Sep. 22, 2021 (Atty Dkt No. 2021PF00227/44755.2210PV01), App. No. 63/246,963, filed Sep. 22, 2021 (Atty Dkt No. 2021PF00228/44755.2212PV01), App. No. 63/246,919, filed Sep. 22, 2021 (Atty Dkt No. 2021PF00225/44755 0.2213PV01), and App. No. 63/246,929, filed Sep. 22, 2021 (Atty Dkt No. 2021PF00226/44755.2214PV01), the entireties of which are incorporated by reference herein.

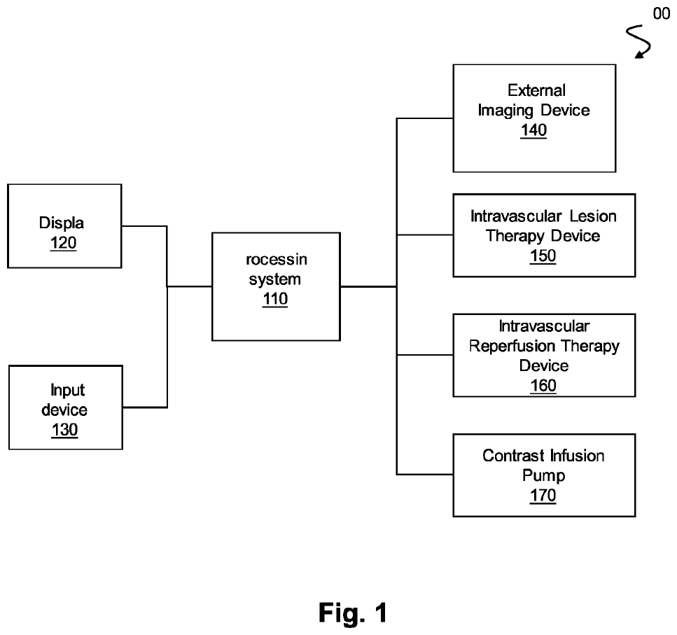

Referring to FIG. 1, shown therein is a system 100 according to an embodiment of the present disclosure. The system 100 can be configured to evaluate (e.g., assess), display, and/or control (e.g., modify) a progress of a reperfusion therapy targeting an area of a patient's body, such as a portion of the myocardium. For instance, the system 100 may be utilized to monitor and/or control reperfusion therapy such that injury to the myocardium following a percutaneous coronary intervention (PCI) is avoided or minimized, as described in greater detail below. In this regard, the system 100 may be used to assess coronary vessels and/or heart tissue (e.g., the myocardium) oxygenated by the coronary vessels. As illustrated, the system 100 includes a processing system 110 in communication with a display 120 (e.g., an electronic display), an input device 130 (e.g., a user input device), an external imaging device 140, an intravascular lesion therapy device 150 (e.g., intraluminal therapy device), an intravascular reperfusion therapy device 160 (e.g., intraluminal reperfusion therapy device), and a contrast infusion pump 170.

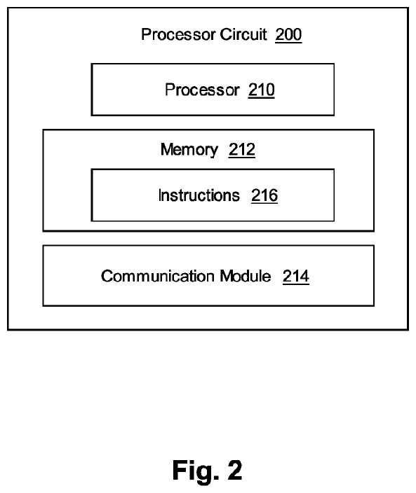

The processing system 110 is generally representative of any device suitable for performing the processing and analysis techniques disclosed herein. In some embodiments, the processing system 110 includes processor circuit, such as the processor circuit 200 of FIG. 2. In some embodiments, the processing system 110 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processor circuit (e.g., computing device) using corresponding instructions stored on or in a non-transitory computer readable medium accessible by the computing device. In some instances, the processing system 110 is a console device. Further, it is understood that in some instances the processing system 110 comprises one or a plurality of computing devices, such as computers, with one or a plurality of processor circuits. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

FIG. 2 is a schematic diagram of a processor circuit 200, according to embodiments of the present disclosure. The processor circuit 200 may be implemented in the processing system 110 of FIG. 1. As shown, the processor circuit 200 may include a processor 210, a memory 212, and a communication module 214. These elements may be in direct or indirect communication with each other, for example via one or more buses.

The processor 210 may include a central processing unit (CPU), a digital signal processor (DSP), an ASIC, a controller, an FPGA, another hardware device, a firmware device, or any combination thereof configured to perform the operations described herein. The processor 210 may also be implemented as a combination of computing devices, e.g., a combination of a DSP and a microprocessor, a plurality of microprocessors, one or more microprocessors in conjunction with a DSP core, or any other such configuration.

The memory 212 may include a cache memory (e.g., a cache memory of the processor 210), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 212 includes a non-transitory computer-readable medium. The memory 212 may store instructions 216. The instructions 216 may include instructions that, when executed by the processor 210, cause the processor 210 to perform the operations described herein with reference to the processing system 110 (FIG. 1). Instructions 216 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 214 can include any electronic circuitry and/or logic circuitry to facilitate direct or indirect communication of data between various components of the processor circuit 200 and/or the processing system 110 (FIG. 1). Additionally or alternatively, the communication module 214 can facilitate communication of data between the processor circuit 200, the display 120 (e.g., a monitor), the input device 130, the external imaging device 140, the intravascular lesion therapy device 150, the intravascular reperfusion therapy device 160, the contrast infusion pump 170, and/or the like. In that regard, the communication module 214 can be an input/output (I/O) device interface, which may facilitate communicative coupling between the processor circuit 200 and (I/O) devices, such as the input device 130. Moreover, the communication module 214 may facilitate wireless and/or wired communication between various elements of the processor circuit 200 and/or the devices and systems of the system 100 using any suitable communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G.

Turning back now to FIG. 1, the external imaging device 140 can include an x-ray system, angiography system, fluoroscopy system, ultrasound system, computed tomography (CT) system, a magnetic resonance imaging (MRI) system, other suitable imaging devices, and/or combinations thereof. The external imaging device 140 may additionally or alternatively include a nuclear medicine imaging device, such as a gamma camera or a single-photon emission computed tomography (SPECT) system, other suitable devices, and/or combinations thereof. The external imaging device 140 can be configured to acquire imaging data of anatomy, such as the heart and blood vessels, while the external imaging device 140 is positioned outside of the body of the patient. The imaging data can be visualized in the form of two-dimensional and/or three-dimensional images of the heart, blood vessel, and/or other anatomy. In some embodiments, the imaging device 140 need not be an external device that is positioned outside the patient body. For example, the imaging device 140 can be an intracardiac echocardiography (ICE) catheter that obtains images while positioned within a heart chamber. In some embodiments, the imaging device 140 may be an external device in that is it is positioned outside of the particular anatomy that is being imaged (e.g., blood vessels and/or heart), but is positioned inside the patient body. For example, the imaging device 140 can be a transesophageal echocardiography (TEE) probe that obtains images while positioned within an esophagus.

Moreover, the external imaging device 140 may obtain images of the heart that are indicative of the health of the cardiac muscle or myocardium. In particular, the external imaging device 140 can be configured to acquire imaging data that illustrates myocardial perfusion (e.g., myocardial perfusion imaging (MPI) data). For example, MPI data can be collected by imaging a radiopharmaceutical agent, such as thallium, in the patient's heart muscle using a SPECT system. Additionally or alternatively, the imaging data may be obtained by imaging a contrast agent, which may be administered to the patient's vasculature manually or via the contrast infusion pump 170, for example. In any case, the imaging data can illustrate vasculature and/or muscle mass with blood flow and/or vasculature and/or muscle mass that lack of blood flow in areas of the heart.

The contrast infusion pump 170 may administer a contrast agent, which may alter an appearance (e.g., a brightness, an intensity, a contrast) of a feature within an external imaging data, such as the external imaging data obtained by the external imaging device 140. In that regard, the contrast infusion pump 170 may be configured to administer, to the patient, a contrast agent that is radiopaque and enhances the visibility of internal fluids or structures within a patient's anatomy. In some embodiments, for example, the contrast agent absorbs external x-rays from an x-ray source, resulting in decreased exposure on an x-ray detector in conjunction with the x-ray source. The contrast agent may be of any suitable material, chemical, or compound and, before administration to the patient, may be a liquid, powder, paste, tablet, or of any other suitable form. For example, the contrast agent may include iodine-based compounds, barium sulfate compounds, gadolinium-based compounds, microbubbles, or any other suitable compounds, which may be included in a solution or suspension, for example, for administration to the patient. In some embodiments, the contrast agent may include carbon dioxide, which may be a gas. In such cases, the contrast agent may decrease absorption of the external x-rays from the x-ray source, when administered. The contrast agent may additionally be referred to as a radiocontrast agent, a contrast dye, a radiocontrast dye, a contrast material, a radiocontrast material, a contrast media, or a radiocontrast media, among other terms. Further, in some embodiments, the contrast infusion pump 170 may be configured to combine or switch between different contrast agents, which may reduce stress on the patient's body. For instance, the contrast infusion pump 170 may administer a first contrast agent for a period of time and may subsequently administer a different, second contrast agent to the patient during an imaging procedure.

The intravascular lesion therapy device 150 may be any form of device, instrument, or probe sized and shaped to be positioned within a vessel. For example, the intravascular lesion therapy device 150 is generally representative of a guide wire, a catheter, or a guide catheter. However, in other embodiments, the intravascular lesion therapy device 150 may take other forms. In that regard, the intravascular lesion therapy device 150 may be a device configured to deliver a PCI therapy to a vessel. In particular, the intravascular lesion therapy device 150 may be an intravascular guidewire or catheter configured to ablate a lesion (e.g., a blockage) within the vessel, deploy a balloon, a stent, and/or drug to a target site within the vessel, and/or the like. That is, for example, the intravascular lesion therapy device 150 may be a stent or balloon delivery device (e.g., an angioplasty device), a thrombectomy device, an atherectomy device, and/or the like. In that regard, the intravascular lesion therapy device 150 may include a coil retriever, an aspiration (e.g., suction) device, and/or the like to assist in the removal of a clot or occlusion from the patient's vessel. In some embodiments, the intravascular lesion therapy device 150 may include a laser, a blade (e.g., knife), a sanding crown, and/or any suitable device that may assist in the cutting, shaving, sanding, vaporizing, and/or removal of atherosclerotic plaque from the patient's vessel. Additionally or alternatively, the intravascular lesion therapy device 150 may be the therapy itself delivered to the vessel. More specifically, the intravascular lesion therapy device 150 may represent a stent or balloon deployed to the vessel, a drug administered intra or extravascularly (e.g., orally), and/or the like. To that end, while the intravascular lesion therapy device 150 is illustrated as being communicatively coupled to the processing system 110, embodiments are not limited thereto.

In some embodiments, the intravascular reperfusion therapy device 160 may be a device, instrument, or probe sized and shaped to be positioned within a vessel. In particular, the intravascular reperfusion therapy device 160 may be a device or instrument configured to control reperfusion of blood flow into a target tissue area (e.g., capillary bed), such as a portion of the myocardium of a patient. In some embodiments, the target tissue area may be an ischemic area and/or an area of tissue that receives reduced blood flow due to a blockage in an associated vessel (e.g., an upstream artery). As described in greater detail below, treatment (e.g., therapy) directed to the vessel associated with the blockage, such as treatment via the intravascular lesion therapy device 150, may reintroduce or increase blood flow to the target tissue area. To reduce or prevent injury to the target tissue area resulting from this increased blood flow, the intravascular reperfusion therapy device 160 may be positioned intravascularly, such as within a coronary blood vessel, and may be configured to regulate blood flow to the target tissue. In some embodiments, the reperfusion therapy can include administration of anti-inflammatory drug(s) or nitric oxide (NO) to the patient. In some embodiments, the reperfusion therapy can include cold fluid that is provided via the arterial side.

In some embodiments, one or more of the external imaging device 140, the intravascular lesion therapy device 150, the intravascular reperfusion therapy device 160, and/or the contrast infusion pump 170, are located proximate one or more of the processing system 110, the display device 120, and/or the input device 130, such as in the same procedure room. In some embodiments, one or more of the external imaging device 140, the intravascular lesion therapy device 150, the intravascular reperfusion therapy device 160, and/or the contrast infusion pump 170 are located spaced from one or more of the processing system 110, the display device 120, and/or the input device 130, such as in different procedure rooms or facilities. For example, the external imaging device 140, the intravascular lesion therapy device 150, the intravascular reperfusion therapy device 160, and/or the contrast infusion pump 170 can be part of different systems that are communicatively coupled. In that regard, the processing system 110 can be configured to acquire the data collected from the components spaced therefrom and process the data as described herein. The external imaging device 140, the intravascular lesion therapy device 150, the intravascular reperfusion therapy device 160, and/or the contrast infusion pump 170 can be configured to transmit the collected data to the processing system 110.

The system 100 includes a display device 120 that is communicatively coupled to the processing system 110. In some embodiments, the display device 120 is a component of the processing system 110, while in other embodiments, the display device 120 is distinct from the processing system 110. In some embodiments, the display device 120 is a monitor integrated in a console device or a standalone monitor (e.g., a flat panel or flat screen monitor). The processing system 110 can be configured to generate a visual display (e.g., screen display) based on imaging data from the external imaging device 140. The processing system 110 can provide (e.g., output) the screen display to the display device 120. To that end, the display device 120 may be configured to output (e.g., display) a two-dimensional image and/or a two-dimensional representation of the heart, blood vessels, and/or other anatomy, which may be included in the screen display. In some embodiments, the display device 120 is configured to output a three-dimensional graphical representation of the heart, blood vessels, and/or other anatomy. For instance, the display device 120 may be a holographic display device configured to output a three-dimensional holographic display of anatomy. Any suitable display device is within the scope of this disclosure, including self-contained monitors, projection/screen systems, head-up display systems, etc. The display device can implement principles based on moving reflective microelectromechanical systems (MEMS), laser plasma, electro-holography, etc. In some embodiments, the display device 120 is implemented as a bedside controller having a touch-screen display as described, for example, in U.S. Provisional Application No. 62/049,265, titled “Bedside Controller for Assessment of Vessels and Associated Devices, Systems, and Methods,” and filed Sep. 11, 2014, the entirety of which is hereby incorporated by reference herein.

The system 100 includes an input device 130 that is communicatively coupled to the processing system 110. The input device 130 may be a peripheral device, such as a touch sensitive pad, a touch-screen, a joy-stick, a keyboard, mouse, trackball, a microphone, an imaging device, and/or the like. In other embodiments, the user interface device is part of the display device 120, which may be a touch-screen display, for example. Moreover, a user may provide an input to the processing system 110 via the input device 130. In particular, the input device 130 may enable a user to control, via inputs to the processing system 110, one or more of the components of the system 100, such as the external imaging device 140, the intravascular lesion therapy device 150, the intravascular reperfusion therapy device 160, the contrast infusion pump 170, or the processing system 110 itself. Additionally or alternatively, the input device 130 may facilitate interaction with a screen display provided at the display device 120. For instance, a user may select, edit, view, or interact with portions of the screen display (e.g., a GUI) provided at the display device 120 via the input device 130.

The system 100 can include various connectors, cables, interfaces, connections, etc., to communicate between the elements of the intravascular lesion therapy device 150, the intravascular reperfusion therapy device 160, the processing system 110, the external imaging device 140, the display device 120, and/or the input device 130. In some embodiments, for example, the communication module 214 (FIG. 2), which may be included in the processing system 110, may include such connectors, interfaces, and/or the like. In that regard, the processing system 110 can communicate and/or control one or more components of the system 110 via mechanical and/or electromechanical signaling and/or controls. Further, the illustrated communication pathways are exemplary in nature and should not be considered limiting in any way. In that regard, it is understood that any communication pathway between the components of system 100 may be utilized, including physical connections (including electrical, optical, and/or fluid connections), wireless connections, and/or combinations thereof. In that regard, it is understood that the one or more of the components of the system 100 can communicate via a wireless connection in some instances. In some instances, the one or more components of the system 100 and/or other systems (e.g., of a hospital or health services provider) communicate via a communication link over a network (e.g., intranet, internet, telecommunications network, and/or other network).

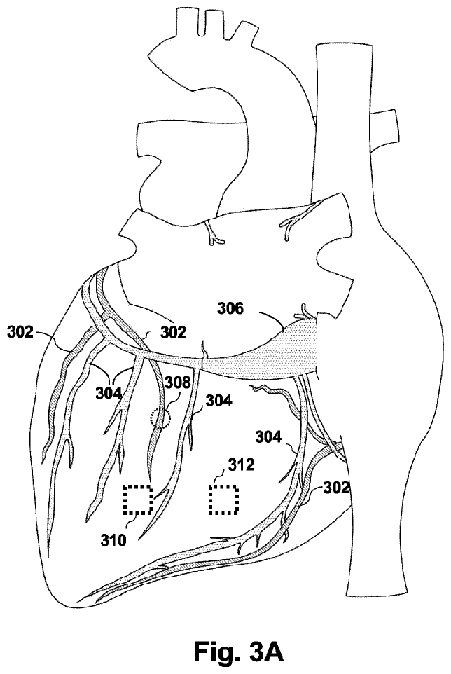

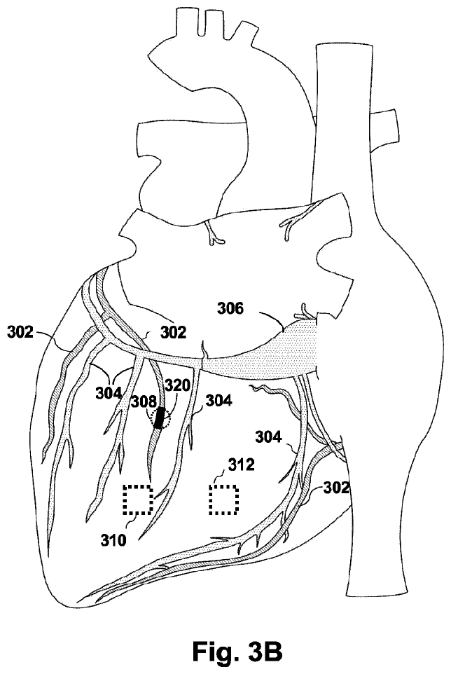

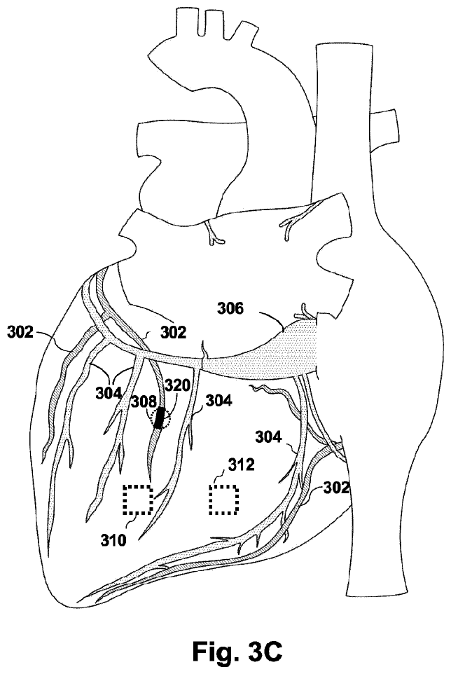

FIGS. 3A-3C illustrate a diagram of a human heart 300. As illustrated, the heart 300 includes coronary arteries 302 (illustrated with a first fill pattern) that deliver oxygenated blood to tissue, such as muscle tissue (e.g., myocardium), of the heart 300. The heart 300 further includes coronary veins 304 (illustrated with a second fill pattern), including a coronary sinus 306, that carry deoxygenated blood away from the tissue of the heart and towards a chamber (e.g., an atrium) of the heart 300.

In the diagram illustrated in FIG. 3A, a coronary artery 302 of the heart 300 includes a blockage 308 (e.g., an occlusion, a lesion, a stenosis, and/or the like). The blockage 308 may disrupt flow through the coronary artery 302. In particular, the blockage 308 may decrease the diameter of a portion of the lumen of the coronary artery 302, which may decrease the flow of blood through the portion of the lumen. As a result, a first area of tissue 310 (e.g., a portion of the myocardium) that is associated with (e.g., receives blood from) the coronary artery 302 with the blockage 308 may not receive a healthy amount of blood/oxygen. For instance, the blood/oxygen delivered to the first area of tissue 310 may not be sufficient to perfuse through (e.g., to be distributed across) the entire first area of tissue 310 in some cases. In this regard, the first area of tissue 310 may experience ischemia (e.g., a reduction in delivered blood/oxygen illustrated by a relatively dark fill pattern), which may damage the first area of tissue 310. In some embodiments, the first area 310 can be and/or include the coronary artery 302 with the blockage. In some embodiments, the first area of the heart may be vasculature and/or tissue associated with (e.g., receiving blood flow from) a first vessel with a previous blockage (as opposed to a current blockage). In some embodiments, the first area may be an area that experiences or has experienced a period of reduced blood flow/ischemia. The illustrated different, second area of tissue 312 (e.g., a portion of the myocardium) may receive blood/oxygen from a different coronary artery 302 than the first area of tissue 310. In this regard, the second area of tissue 312 may remain relatively unaffected by the blockage 308. To that end, the second area of tissue 312 may receive a healthy amount of blood/oxygen, and the second area of tissue 312 may not experience ischemia. Accordingly, the second area of tissue 312 is illustrated as being healthy by a lack of the fill pattern (e.g., by a relatively brighter and/or whiter fill) shown in the first area of tissue 310. In some embodiments, the second area 312 can be and/or include coronary artery 302 without the blockage.

In some embodiments, a percutaneous coronary intervention (PCI) may be utilized to treat the blockage 308. In particular, the PCI may include a therapeutic procedure that reduces a size of the blockage 308, opens (e.g., widens) the lumen of a vessel, and/or the like to restore blood flow through the vessel (e.g., the coronary artery 302) with the blockage 308. In that regard, the PCI may include, for example, angioplasty (e.g., deploying a balloon) and positioning a stent across the stenosis to open the vessel (e.g., the coronary artery 302 with the blockage). The PCI may additionally or alternatively include thrombectomy, atherectomy, administration of a drug and/or the like. To that end, the intravascular lesion therapy device 150 (FIG. 1) may facilitate and/or provide the PCI to a vessel having a blockage (e.g., blockage 308).

FIG. 3B illustrates a diagram of the heart 300 after delivery of a therapeutic procedure (e.g., post-treatment), such as PCI. In particular, FIG. 3B illustrates a stent 320 positioned within the coronary vessel at the site of the blockage 308. However, embodiments are not limited thereto. In that regard, the PCI delivered to the coronary artery 302 or a vessel with a blockage may include any suitable combination of the therapies described above.

As described above, the stent 320 and/or another suitable PCI (e.g., therapeutic procedure) may be provided to a vessel so that an effect of a blockage on blood flow through the vessel is reduced. In this regard, the placement of the stent 320 within the coronary artery 302 (e.g., at the site of the blockage 308) may open (e.g., widen) the portion of the lumen of the coronary artery 302 with the blockage 308, which may increase blood flow through the portion lumen. Moreover, the placement of the stent 320 within the heart 300 may increase blood flow downstream of the blockage 308, such as within vasculature that receives blood flow from the portion of the lumen. In this way, the vasculature (e.g., a capillary bed) that delivers blood/oxygen to the first area of tissue 310 may receive increased blood flow, which may increase blood/oxygen delivery to the first area of the tissue 310. To that end, blood/oxygen may re-perfuse the first area of the tissue 310. Accordingly, the stent 320 may reverse or reduce the ischemia experienced by the first area of the tissue 310. In this regard, the first area of the tissue 310 is illustrated in FIG. 3B with a different fill pattern (e.g., a relatively brighter and/or lighter fill pattern) than the fill pattern illustrated in FIG. 3A to demonstrate the increased blood/oxygen supplied to the first area of the tissue 310.

In some cases, blood/oxygen may not suitably perfuse through tissue associated with an occluded vessel (e.g., a vessel with a blockage), such as the first area of tissue 310, after delivery of a PCI therapy. For example, in some cases, the introduction and/or increase of blood flow to tissue that has experienced ischemia may result in reperfusion injury (e.g., ischemia-reperfusion injury). In particular, the returned blood flow may trigger an inflammatory response and/or oxidative damage along with or in place of restoration of normal function of the tissue. Inflammation, damage resulting from inflammation, and/or the oxidative damage may obstruct the flow of blood/oxygen within the tissue (e.g., within a capillary bed associated with the tissue). Accordingly, blood/oxygen may not be distributed throughout (e.g., perfuse through) the tissue at a healthy level even after the delivery of a PCI therapy. For instance, blood may preferentially flow through a first portion of the tissue lacking inflammation and/or damage and may flow through a second portion of the tissue with inflammation and/or damage to a lesser degree. As a result, the second portion of the tissue may continue to receive blood flow below a healthy level. In this regard, the first area of the tissue 310 is illustrated in FIG. 3B with a different fill pattern (e.g., a relatively darker fill pattern) than the second area of the tissue 312 (e.g., a healthy area of tissue) to demonstrate that the stent 320 alone may not fully restore the health and/or functioning of the first area of the tissue 310.

Turning now to FIG. 3C, in some cases, the reperfusion of blood/oxygen within ischemic tissue may be further assisted and/or controlled by reperfusion therapy. To that end, FIG. 3C illustrates a diagram of the heart 300 after delivery of the PCI therapy and a reperfusion therapy, such as a therapy delivered by the intravascular reperfusion therapy device 160. In particular, FIG. 3C illustrates a diagram of the heart 300 following delivery of a reperfusion therapy targeting the first area of tissue 310. According to techniques described in greater detail below, reperfusion therapy may be delivered by the intravascular reperfusion therapy device 160 to reduce or minimize injury at and/or to improve blood flow to tissue where blood is re-perfusing (e.g., an area of tissue receiving an increase in blood flow), such as the first area of tissue 310. In particular, reperfusion therapy may affect a distribution of blood flow through the targeted tissue such that blood flow perfuses (e.g., distributes to) and/or increases throughout the tissue, including through areas of the tissue that are inflamed or have oxidative damage. Accordingly, delivery of the reperfusion therapy targeting an area of tissue may restore blood flow to a healthy amount or an amount exceeding the blood flow resulting from the PCI therapy alone. For instance, in the illustrated embodiment, the health and/or functioning of (e.g., the blood flow to) the first area of tissue 310 is shown as being fully restored by the reperfusion therapy, as indicated by the fill pattern of the first area of tissue 310 matching the second area of tissue 312. In some embodiments, however, the reperfusion therapy may restore the health and/or functioning of (e.g., the blood flow to) tissue to a level greater than a level resulting from the PCI but less than a level at an area of tissue, such as the second area of tissue 312, that was relatively unaffected by a blockage (e.g., associated with a different vessel than the vessel having the blockage). Particular mechanisms for controlling, using one or more components of the system 100, the reperfusion of an area of tissue associated with (e.g., configured to receive blood/oxygen from) a vessel that receives a PCI therapy (e.g., a vessel that has an occlusion) and/or otherwise receives an increase in blood flow are described herein.

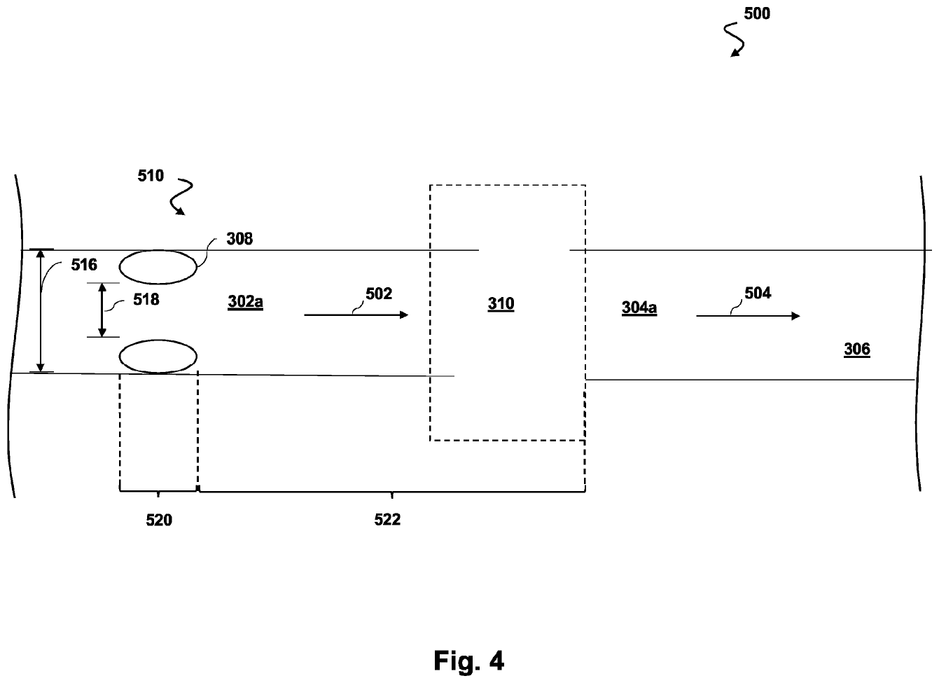

FIG. 4 illustrates a schematic diagram of a portion 500 of a heart of a patient. FIG. 4 may correspond to a detailed view of a portion of the heart 300 illustrated in FIG. 3A. More specifically, FIG. 4 illustrates a coronary artery 302, which includes a blockage 308 (e.g., a stenosis) and is arranged to provide blood/oxygen to an area of tissue 310, as indicated by the arrow 502. A coronary vein 304 is arranged to carry deoxygenated blood away from the area of tissue 310, as indicated by the arrow 504, and is illustrated as being in fluid communication with the coronary sinus 306.

As described above with respect to FIGS. 3A-3B, the blockage 308 may disrupt blood flow through the coronary artery 302. In particular, the blockage 308 may reduce a diameter of a lumen of the coronary artery 302 from a first diameter 516 to a smaller, second diameter 518 within the portion 520 of the vessel where the blockage 308 is located. This reduction in diameter may increase the resistance and/or the impedance to blood flowing through the portion 520, which may reduce blood/oxygen delivered distal of the blockage (e.g., within the distal area 522 including a portion of the coronary artery 302 and the area of tissue 310). In this regard, the area of tissue 310 (e.g., a portion of myocardium) may experience ischemia (e.g., a reduction in delivered blood/oxygen), which may damage the area of tissue 310.

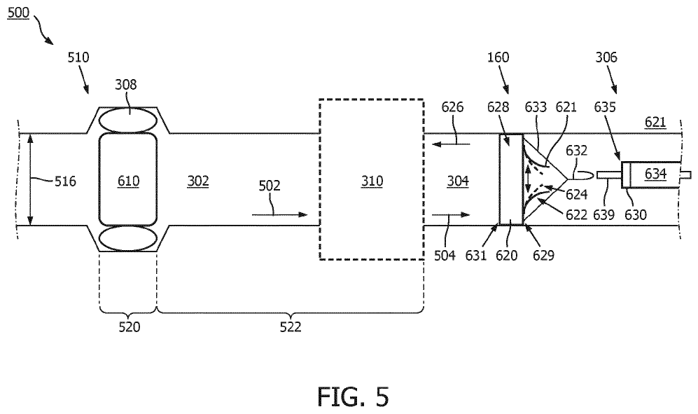

FIG. 5 is a schematic diagram of the portion 500 of the heart during and/or following delivery of a PCI therapy 610 and/or during delivery of a reperfusion therapy. FIG. 5 may correspond to a detailed view of a portion of the heart 300 illustrated in FIG. 3B. The PCI therapy 610 may be delivered by the intravascular lesion therapy device 150 and/or the PCI therapy 610 may be the intravascular lesion therapy device 150 itself. For instance, as described above with respect to FIG. 1, the PCI therapy 610 may involve ablation of the blockage 308, deployment of a balloon (e.g., angioplasty), stent, and/or drug, thrombectomy, atherectomy, and/or the like. In that regard, the PCI therapy 610 may involve a therapeutic procedure that widens the diameter available for blood flow in the portion 520 having the blockage 308 from the diameter 518 (FIG. 4). That is, for example, the PCI therapy 610 may involve widening the diameter of the lumen of the coronary artery 302, as shown. Additionally or alternatively, a size of the blockage 308 may be reduced (e.g., ablated or removed) to increase the diameter available for blood flow within the portion 520. Further, the PCI therapy 610 may involve the placement of a physical device, such as a stent, within the coronary artery 302. In some embodiments, the PCI therapy 610 may involve the use of a device (e.g., a guidewire or catheter) that is removed from the coronary artery 302 when the PCI therapy 610 is completed. In that regard, the illustrated representation of the PCI therapy 610 positioned within the coronary artery 302 is intended to be exemplary and not limiting.

FIG. 5 further illustrates the intravascular reperfusion therapy device 160, which is shown as including or being associated with (e.g., controlling and/or deploying) a stent 620 that includes a valve 621 (e.g., a valve stent). To that end, while the intravascular reperfusion therapy device 160 and the stent 620 are referred to separately, it may be appreciated that the stent 620 may be a component of the intravascular reperfusion therapy device 160. In some embodiments, the intravascular reperfusion therapy device 160 may deliver reperfusion therapy targeting (e.g., in association with) the area of tissue 310. In that regard, the stent 620 is shown as being positioned at and delivering reperfusion therapy via a venous side (e.g., within the coronary sinus 306) of the area of tissue 310.

In some embodiments, reperfusion therapy delivered to a venous side of the area of tissue 310 may involve obstruction of vasculature on the venous side of the first area of tissue 310. In the illustrated embodiment, for example, the stent 620 is arranged to obstruct blood flow through a coronary vein 304 (e.g., the coronary sinus 306). More specifically, the stent 620 includes a valve 621 (e.g., a pressure-regulating orifice) arranged to obstruct (e.g., restrict) blood flow through the coronary vein 304. In particular, the valve 621 transitions (e.g., reduces) a dimension (e.g., a diameter and/or an area) of the stent 620 available for blood flow from a first dimension to a second, smaller dimension. By obstructing the vasculature at the valve 621, the stent 620 may increase pressure on the venous side of the tissue, which may encourage better (e.g., increased and/or more even) distribution of blood flow through the area of tissue 310, especially the damaged or inflamed portions of the area of tissue 310. In that regard, the valve 621 is shown as delivering reperfusion therapy by generating a back pressure and/or back flow in a direction indicated by arrow 626 via restriction of blood flow through the coronary vein in the opposite direction, indicated by arrow 504. As described in greater detail below, the back pressure and associated reperfusion therapy supplied by the stent 620 may be controlled via control of a configuration of the valve 621 and/or control of an expansion state of the stent 620.

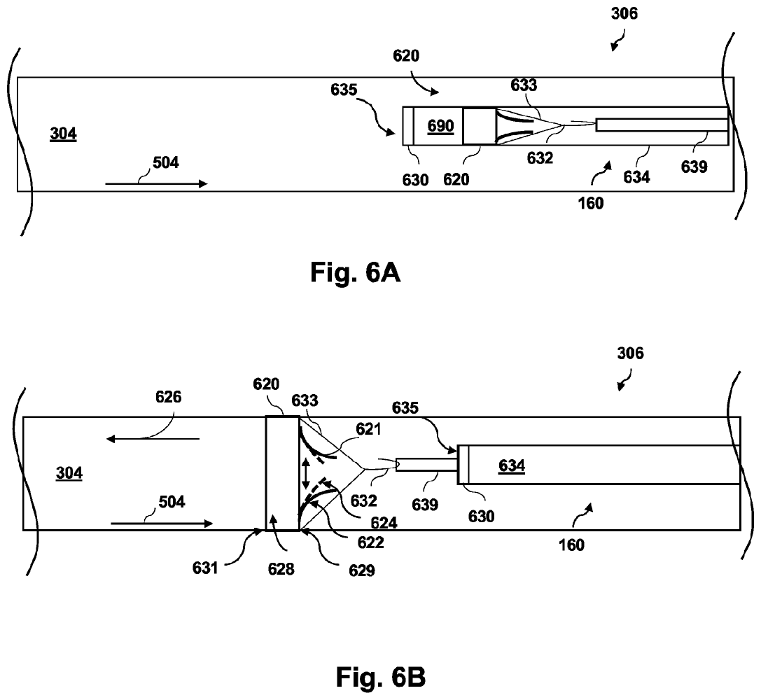

As further illustrated, the intravascular reperfusion therapy device 160 may include one or more flexible elongate members, such as a catheter 634 and/or a guidewire, which may extend through a portion of the catheter 634. In some embodiments, the stent 620 may be deployed from and/or retracted (e.g., retrieved) into a lumen (e.g., a hollow interior) of the catheter 634, as described with respect to FIGS. 6A-6B. The intravascular reperfusion therapy device 160 may further include one or more sensors (e.g., sensing components). For instance, a sensor 630 may be positioned at the distal portion of the catheter 634, such as at a distal end of the catheter 634 or spaced from and adjacent to the distal end of the catheter 634.

While a catheter 634 is illustrated, embodiments are not limited thereto. In some embodiments, for example, the intravascular reperfusion therapy device 160 may be implemented with the catheter 634 and a guidewire or with a guidewire and no catheter 634. Similarly, the illustrated embodiment including the sensor 630 is intended to be illustrative and not limiting. In that regard, the intravascular reperfusion therapy device 160 may be implemented with any suitable number of sensors, such as a single sensor, two, three, four sensors, and/or the like.

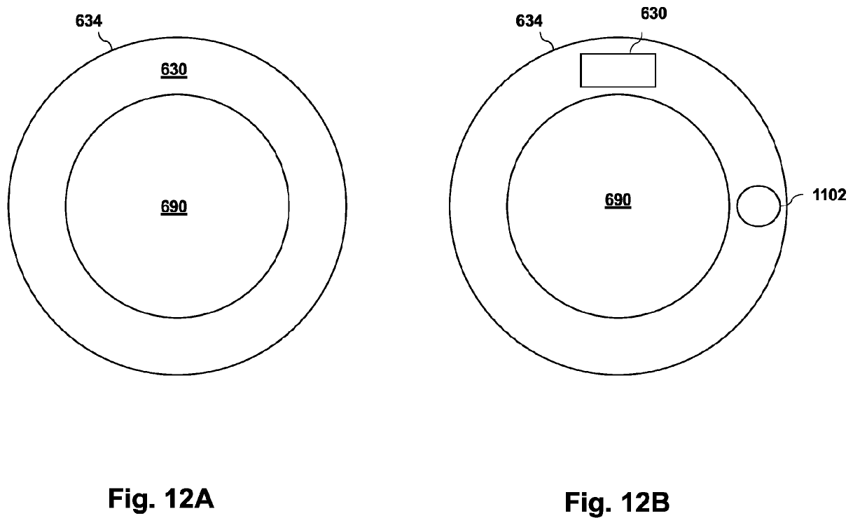

In some embodiments, the sensor 630 of the intravascular reperfusion therapy device 160 may be configured to sense (e.g., obtain) physiological data from within a blood vessel of the patient, such as a coronary vein 304 and/or the coronary sinus 306. In that regard, the physiological data may be associated with blood flow through the blood vessel. For instance, the sensor 630 may obtain flow data associated with a flow rate or flow velocity of blood through the blood vessel, pressure data associated with a blood pressure within the blood vessel, and/or the like. To that end, the sensor 630 may include a flow sensor configured to obtain the flow data, a pressure sensor configured to obtain the pressure data, and/or the like. In some embodiments, the intravascular reperfusion therapy device 160 may additionally or alternatively include an imaging device configured to obtain intravascular imaging data, such as intravascular ultrasound (IVUS) imaging data, optical coherence tomography (OCT) imaging data, and/or the like. The imaging data may be used to determine pressure data, flow data, and/or characteristics of the blood vessel, such as a diameter of the vessel, which may be useful for determining the pressure and/or flow data. Additionally or alternatively, the intravascular reperfusion therapy device 160 may include a sensor configured to determine and/or monitor a configuration of the valve 621 such that a level of obstruction of the blood vessel may be determined and/or monitored. In some embodiments, the intravascular reperfusion therapy device 160 may include one or more sensors that are the same or different from one another. As an illustrative example, the sensor 630 may be a flow sensor, while an additional sensor may be a pressure sensor or vice versa. As another example, the sensor 630 and the additional sensor may each be pressure sensors or may each be flow sensors. Details of the configuration of the one or more sensors are provided below with respect to FIGS. 12A-12B.

The stent 620 may include the valve 621, a stent body 620, and a retrieval hook 632 (e.g., a snare or retrieval component). The stent body 628 may be a tube or tube-like structure, which may expand to contact a wall of the coronary vein 304. For instance, the stent body 628 may be formed from a wire mesh, a self-expanding (e.g., shape-memory) material, and/or the like. By expanding into contact with the wall of the coronary vein 304, the stent body 628 may anchor the stent 620 in place (e.g., at a fixed position) within the vasculature. The stent body 628 may further be coupled to the valve 621 and the retrieval hook 632. In some embodiments, the valve 621 and the retrieval hook 632 may be coupled to a proximal end 629 of the stent body 628, as illustrated. In some embodiments, the valve 621 may be coupled to a distal end 631 of the stent body 628, while the retrieval hook 632 is coupled to the proximal end 629 of the stent body 628. In some embodiments, the retrieval hook 632 may be coupled to the distal end 631 of the stent body 628.

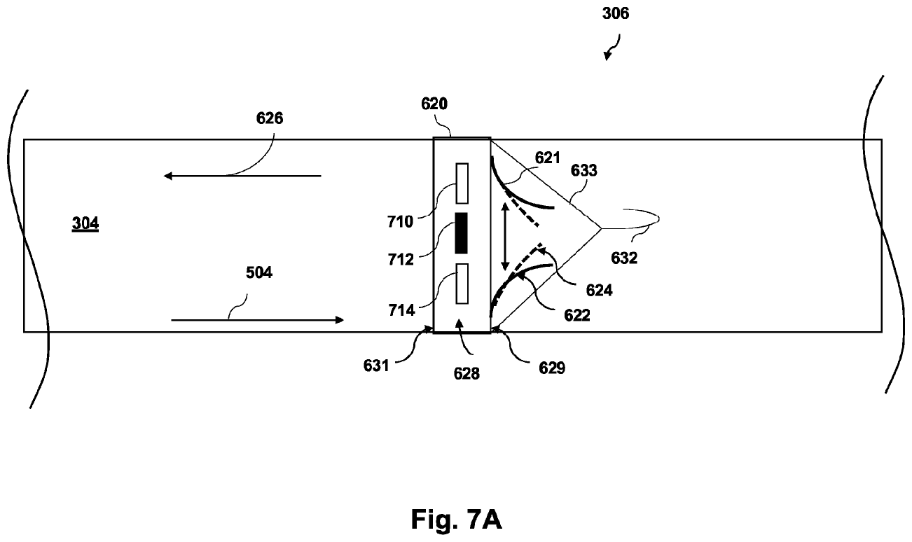

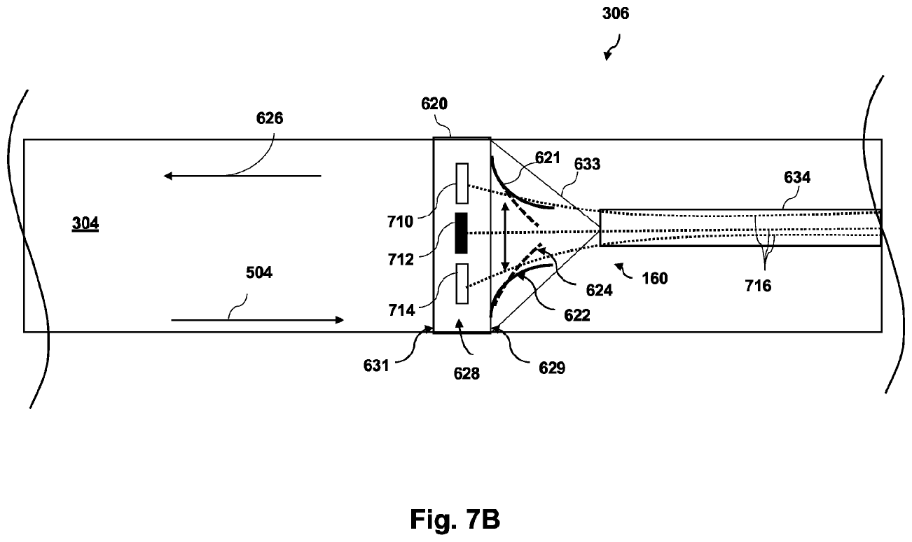

In some embodiments, once anchored within a blood vessel (e.g., via the stent body 628), the stent 620 may remain implanted for a duration of at least a portion of a reperfusion therapy. For instance, the stent 620 may remain implanted over a period of one or more hours, days, and/or the like and may deliver the reperfusion therapy over this period. Moreover, in some embodiments, the implanted stent may passively deliver (e.g., without control via a processor circuit 200 and/or the processing system 110) the reperfusion therapy over at least a portion of this period. For instance, the valve 621 may remain in a fixed configuration or a series of configurations, as altered by the blood flow, in the vasculature over this period. In some embodiments, the implanted stent 620 may actively deliver the therapy over at least a portion of this period. For instance, the valve 621 may change configurations under the control of (e.g., responsive to a signal from) a processor circuit 200, such as a processor circuit 200 associated with the processing system 110 and/or a processor circuit 200 associated with the sensor itself, as described with respect to FIGS. 7A and 7B.

For implantation in the patient's vasculature, the stent 620 may be removably coupled from the intravascular reperfusion therapy device 160 (e.g., from a component of the catheter 634). To that end, the catheter 634 may be used to deploy the stent 620 within a blood vessel, and, in some embodiments, may then retrieve the stent 620 from the patient's vasculature. In some embodiments, such as cases where the reperfusion therapy is delivered over a relatively short time span, for example, the catheter 634 may remain positioned within the patient's vasculature after the stent 620 is deployed. In some embodiments, the catheter 634 may be removed from the patient's vasculature when the stent 620 is deployed and may subsequently be reintroduced into the vasculature to retrieve the stent 620. In any case, the catheter 634 may retrieve a deployed stent 620 from within the blood vessel. Further, in some embodiments, delivery of a reperfusion therapy may be terminated via the retrieval of the stent 620 with the catheter 634.

In some embodiments, the catheter 634 may control the deployment and/or retrieval of the stent 620 via interaction of an actuator 639 with the retrieval hook 632. In particular, the catheter 634 may deploy and/or retrieve the stent 620 at a distal end 635 of the catheter 634. Accordingly, in embodiments where the retrieval hook 632 is coupled to the distal end 631 of the stent body 628, a portion of the retrieval hook 632 may extend proximally through the stent body 628 such that the catheter 634 may retrieve the stent 620. Moreover, the interaction of the actuator 639 with the retrieval hook 632 may involve a mechanical interaction, such as the exertion of a mechanical force by the actuator on the retrieval hook 632 to push or pull the stent 620 into a particular position. Further, in some embodiments, the stent 620 may be removably coupled to the actuator 639 via a mechanical coupling, which may involve the retrieval hook 632 latching or hooking onto a portion of the actuator 639, a magnetic (e.g., electromagnetic) coupling, and/or the like. To that end, deploying the stent 620 may involve the actuator 639 mechanically, electromechanically, or electromagnetically releasing the retrieval hook 632, and retrieving the stent 620 may involve the actuator 639 mechanically, electromechanically, or electromagnetically engaging the retrieval hook 632. In some embodiments, the stent 620 may remain coupled to the actuator 639 after deployment (e.g., after being positioned distal of the distal end 635 of the catheter 634). For instance, the stent 620 may be fixedly coupled to the actuator 639 or the actuator 639 may be controlled such that the retrieval hook 632 remains engaged with the actuator 639. Moreover, in some embodiments, the stent 620 may be deployed from a circumferential position about the catheter 634. For instance, the catheter 634 may include a balloon configured to expand to deploy the stent 620. Further, in some embodiments, the catheter 634 itself may engage with the stent 620 (e.g., via the retrieval hook 632) to deploy or retrieve the stent 620. Thus, in some embodiments, the intravascular reperfusion therapy device 160 may lack the actuator 639. Accordingly, while the actuator 639 is illustrated as deploying or retrieving the stent 620 by extending distal of the distal end 635, embodiments are not limited thereto.

As described, the valve 621 may transition (e.g., reduce) the diameter of the stent 620. In that regard, the valve 621 may be a windsock valve, a duckbill valve, or any other suitable valve with a reduction, such as a taper, in diameter. Further, in some embodiments, the valve 621 may include one or more configurations, such as the illustrated first configuration 622 and second configuration 624, which may each provide a respective level (e.g., degree) of obstruction within a blood vessel. For instance, the first configuration 622 provides a first diameter of the stent 620 (e.g., diameter within the blood vessel) available for blood flow, causing a first degree of obstruction, while the second configuration 624 provides a second diameter of the stent 620, causing a second, greater degree of obstruction. Because the different configurations (e.g., diameters) of the valve 621 correspond to different degrees of obstruction, the configuration of the valve 621 corresponds to an amount of back pressure generated in the direction (indicated by arrow 626) opposite the direction (indicated by arrow 504) of blood flow. In that regard, the configuration (e.g., diameter) of the valve 621 may correspond to a level (e.g., degree) of reperfusion therapy and/or the aggressiveness of the reperfusion therapy delivered to a target area of tissue (e.g., the area of tissue 310). Moreover, while the valve 621 is illustrated as being arranged in the first configuration 622 and the second configuration 624, it may be appreciated that the valve 621 may be configured to transition between any suitable number of configurations, which may provide a corresponding number of different degrees of obstruction of the vasculature. To that end, the obstruction of the vasculature and resulting reperfusion therapy may be coarsely or finely controlled based on the relative differences between the different configurations of the valve 621.

In some embodiments, the valve 621 may be formed with a material and/or a configuration that is deformable (e.g., adjustable). That is, for example, the valve 621 may be formed from a material configured to change shape or configurations in response to an electrical signal, thermal energy, pressure (e.g., a mechanical force), and/or the like. As an illustrative example, the valve 621 may be formed from an electroactive polymer (EAP), which may exhibit a shape change in response to an electrical signal. As an additional example, the valve 621 may be formed as a bimetallic strip or other suitable material configured to hold a first shape (e.g., configuration) for a first range of temperatures and to transition to a different, second shape (e.g., configuration) for a different, second range of temperatures (e.g., responsive to the application or dissipation of thermal energy). As another example, the valve 621 may be formed from an elastic material configured to bend from a first shape to a second shape when a pressure is applied to the valve 621 and/or the stent 620 and to return to the first shape when the pressure is no longer applied to the valve 621. Further, in some embodiments, the valve 621 may additionally or alternatively be formed with joints or other features suitable to alter the shape of the valve 621 responsive to an electrical signal, the application or dissipation of thermal energy, and/or a mechanical force. In some embodiments, the valve 621 may be formed from a variety of materials that may be deformed according to the techniques described above, including, by way of non-limiting example, plastics, polytetrafluoroethylene (PTFE), polyether block amide (PEBAX), thermoplastic, polyimide, silicone, elastomer, polymers, resins, carbon fiber, metals, such as stainless steel, titanium, superelastic or shape-memory alloys such as Nitinol, composite materials, and/or other biologically compatible materials.

In some embodiments, the intravascular reperfusion therapy device 160 may control the configuration of the valve 621. For instance, in some embodiments, the intravascular reperfusion therapy device 160 may supply an electrical signal, a mechanical force, and/or thermal energy to the stent 620 to control the configuration of the valve 621. In some embodiments, for example, the actuator 639 may be a mechanical actuator arranged to provide a mechanical force at the valve 621 via interaction with the retrieval hook 632 or another portion of the stent 620 (e.g., the valve 621 itself). In some embodiments, the actuator 639 may be an electromechanical actuator arranged to provide the electrical signal to the valve 621. Further, as described above, the valve 621 may be arranged to change configurations responsive to this electrical signal. For instance, the valve 621 may be constructed with an electroactive polymer that is responsive to the electrical signal. Additionally or alternatively, the actuator 639 may provide the thermal energy to the valve 621 via a heating or cooling system. For instance, the actuator 639 may include a conduit (e.g., tubing) that circulates a cooling fluid to affect a temperature change and, as a result, a configuration change at the valve 621. Additionally or alternatively, the actuator 639 may be in communication with a heat pump, which may affect a temperature change at the valve 621 via the actuator 639. Accordingly, in some embodiments, the configuration of the valve 621 may be controlled via a component of the catheter 634 (e.g., the actuator 639). For instance, the actuator 639 may remain in contact with the deployed stent 620, as described above, and may deliver the mechanical force, electrical signal, or thermal energy via this coupling. In some embodiments, the configuration of the valve 621 may be controlled remotely (e.g., wirelessly). In particular, the stent 620 may be configured to receive a signal to adjust the configuration of the valve 621 and may control the configuration of the valve based on this signal, as described with respect to FIGS. 7A and 7B. Moreover, in some embodiments, flow of blood through the blood vessel may supply a mechanical force sufficient to deform (e.g., adjust the configuration of) the valve 621.

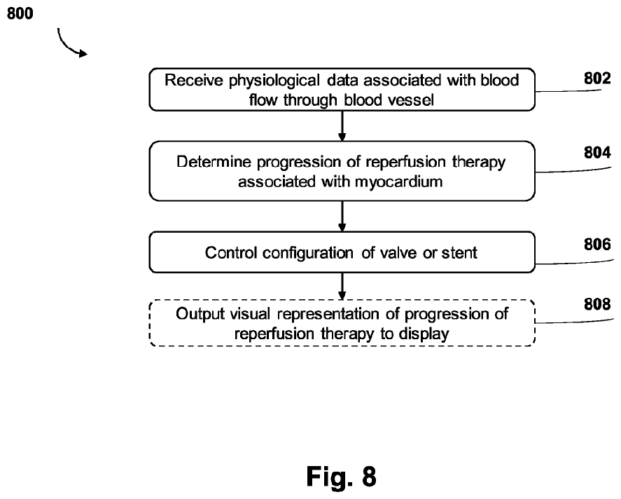

In some embodiments, the valve 621 may be oscillated between different configurations, to deliver reperfusion therapy. For instance, the valve 621 may be oscillated between the first configuration 622, which may obstruct the vasculature to a first degree, and the second configuration 624, which may obstruct the vasculature to a second, greater degree (e.g., more fully obstruct the vasculature). In some embodiments, the intravascular reperfusion therapy device 160 and/or the processing system 110 may be configured to oscillate the valve 621 according to a set pattern (e.g., combination of frequency, duty cycle, duration, and/or the like). For instance, the processing system 110 may include instructions (e.g., instructions 216) to deliver a predetermined reperfusion therapy via a set (e.g., predetermined) control (e.g., oscillation) of the valve 621 between configurations. Additionally or alternatively, the processing system 110 may be configured to control the delivery of the reperfusion therapy (e.g., via control of configuration of the valve 621) based on a user input, which may be received via the input device 130, for example. Moreover, in some embodiments, the processing system 110 may be configured to monitor a progression of the reperfusion therapy and adaptively control the delivery of the reperfusion therapy based on the progression of the reperfusion therapy. For instance, as described with respect to FIG. 8, the processing system 110 may control the delivery of the reperfusion therapy based on information (e.g., physiological data) received from the one or more sensors (e.g., the sensor 630 and/or an additional sensor) of the intravascular reperfusion therapy device 160. That is, for example, the processing system 110 may control delivery of the therapy via a feedback loop with the intravascular reperfusion therapy device 160.

FIGS. 6A-B illustrate an exemplary intravascular reperfusion therapy device 160 that includes one or more sensors (e.g., 630) and is arranged to deploy and/or control a stent 620 with a valve 621. In particular, FIG. 6A is a diagrammatic cross-sectional side view of the intravascular reperfusion therapy device 160 with the stent 620 in an undeployed, collapsed state (e.g., a first expansion state), and FIG. 6B is a diagrammatic side view of the intravascular reperfusion therapy device 160 with the stent 620 in a deployed, expanded state (e.g., a second expansion state).

FIG. 6A illustrates the stent 620 positioned within a lumen 690 of the catheter 634 and arranged in a collapsed state (e.g., a first expansion state). In some embodiments, expansion of the stent 620 may be restrained (e.g., limited) by the diameter of the lumen 690 such that the stent 620 remain in the collapsed state while positioned within the catheter 634. As further shown, the stent 620 may be coupled to the actuator 639, which may be a flexible elongate member, and may also be positioned within the lumen 690. In some embodiments, the retrieval hook 632 of the stent 620 may be coupled to the actuator 639. Further, in some embodiments, the actuator 639 may extend to a proximal portion of the intravascular reperfusion therapy device 160, where the positioning of the actuator 639 and the stent 620 may be manually or automatedly controlled. For instance, the actuator 639 may be coupled to one or more knobs, gears, wires, telescoping portions, and/or the like at the proximal portion, which may be controlled by a physician or the processing system 110.

In some embodiments, the stent 620 may be positioned in the illustrated undeployed, collapsed state before the stent 620 is introduced into a blood vessel of a patient (e.g., before a reperfusion therapy is initiated). The stent 620 may additionally or alternatively be positioned in the illustrated position within the lumen 690 after the stent 620 is retrieved from the blood vessel (e.g., after or during delivery of the reperfusion therapy). To that end, the retrieval hook 632 of the stent 620 may be arranged such that, responsive to a mechanical force (e.g., a pulling) on the retrieval hook 632, the stent body 628 transitions the stent 620 to the collapsed state. For instance, the retrieval hook may include a snare 633 that collapses the stent body 628 responsive to the mechanical force. In particular, portions of the snare 633 may be brought in closer proximity to one another responsive to the mechanical force, which may cause portions of the stent body 628 coupled to the portions of the snare 633 to be collapsed into closer proximity to one another as well. Further, the mechanical force may be supplied by the actuator 639 pulling the stent 620 into the catheter 634, for example. The stent 620 may additionally or alternatively be retrieved and/or transitioned to the collapsed state using an electrical signal, magnetism, and/or the like, which may be supplied by the intravascular reperfusion therapy device 160 (e.g., via the actuator 339). Moreover, while the stent 620 is illustrated as being positioned within the lumen 690 in an undeployed state, embodiments are not limited thereto. In that regard, the stent 620 may be positioned about a perimeter (e.g., a circumference) of the catheter 634 in the undeployed state in some embodiments.

FIG. 6B illustrates the stent 620 in an expanded state (e.g., a second expansion state). As illustrated, in the expanded state, the stent 620 may expand within the blood vessel (e.g., a coronary vein 304). In particular, the stent body 628 may expand to a diameter 814, which may substantially match a diameter of the blood vessel such that the stent is anchored (e.g., coupled) to a wall of the blood vessel. In some embodiments, the stent body 628 may be formed from a material and/or with a configuration that enables the stent body 628 to transition between the collapsed state shown in FIG. 6A and the expanded state shown in FIG. 6B. For instance, the stent body 628 may be formed from a wire mesh, a self-expanding material, and/or the like. More specifically, the stent body 628 may be formed from one or more plastics, polytetrafluoroethylene (PTFE), polyether block amide (PEBAX), thermoplastic, polyimide, silicone, elastomer, polymers, resins, carbon fiber, metals, such as stainless steel, titanium, superelastic or shape-memory alloys such as Nitinol, composite materials, and/or other biologically compatible materials.

In some embodiments, the stent 620 may manually (e.g., via user control) or automatedly (e.g., via control by the processing system 110) transition from the collapsed state to the expanded state or vice versa. For instance, the processing system 110 may control a position (e.g., actuate) of the actuator 639 so that the stent 620 is positioned within or distal of the catheter 634. In some embodiments, such as cases where the lumen 690 limits the diameter of the valve 621, the stent 620 may be arranged to expand once the stent 620 is spaced from the catheter 634. To that end, the stent 620 may relax to the expanded state after leaving the confines of the catheter 634. Additionally or alternatively, the processing system 110 may control an electrical signal or a thermal energy applied to the valve 621 to transition the stent 620 between expansion states. Moreover, the processing system 110 may be configured to oscillate the stent 620 between the collapsed and expanded states to control delivery of reperfusion therapy based on physiological data obtained from the sensor 630. For instance, the stent 620 may not obstruct the vasculature in the collapsed state or may obstruct the vasculature to a relatively low degree, and the stent 620 may obstruct the vasculature to a greater degree in the expanded state (e.g., via the valve 621). To that end, the expansion state of the stent 620 may correspond to the amount of back pressure generated in the direction (indicated by arrow 626) opposite the direction (indicated by arrow 504) of blood flow. Accordingly, by controlling the intravascular reperfusion therapy device 160 to deploy or retrieve the stent 620, the processing system 110 may control the expansion state of the stent 620 and the resulting delivery of reperfusion therapy. Moreover, while the stent 620 is illustrated as being arranged in the expanded state or the collapsed state, it may be appreciated that the stent 620 may be configured to expand according to any number of degrees of expansion. For instance, in some embodiments, the intravascular reperfusion therapy device 160 may maintain the stent 620 in a partially expanded state. To that end, the embodiments described herein are intended to be exemplary and not limiting.

In some embodiments, the stent 620 may be oscillated between different expansion states to deliver reperfusion therapy. For instance, the stent 620 may be oscillated between the expanded state illustrated in FIG. 6B, which may obstruct the vasculature via the valve 621 to a first degree or range of degrees, and the collapsed state illustrated in FIG. 6A, which may not obstruct the vasculature or may obstruct the vasculature to a second degree less than the first degree and range of degrees. In particular, the stent 620 may be oscillated between different expansion states via deployment and retrieval of the stent 620. In some embodiments, the intravascular reperfusion therapy device 160 and/or the processing system 110 may be configured to oscillate the stent 620 according to a set pattern (e.g., combination of frequency, duty cycle, duration, and/or the like). For instance, the processing system 110 may include instructions (e.g., instructions 216) to deliver a predetermined reperfusion therapy via a set (e.g., predetermined) control (e.g., oscillation) of the stent 620. Additionally or alternatively, the processing system 110 may be configured to control the delivery of the reperfusion therapy (e.g., via control of expansion of the stent 620) based on a user input, which may be received via the input device 130, for example. Moreover, in some embodiments, the processing system 110 may be configured to monitor a progression of the reperfusion therapy and adaptively control the delivery of the reperfusion therapy based on the progression of the reperfusion therapy. For instance, as described with respect to FIG. 8, the processing system 110 may control the delivery of the reperfusion therapy based on information (e.g., physiological data) received from the one or more sensors (e.g., the sensor 630 and/or an additional sensor) of the intravascular reperfusion therapy device 160. That is, for example, the processing system 110 may control delivery of the therapy via a feedback loop with the intravascular reperfusion therapy device 160.

FIG. 7A is a diagrammatic side view of an exemplary stent 620 that includes a valve 621. In the illustrated embodiment, the stent 620 is implanted (e.g., anchored) within the blood vessel (e.g., the coronary sinus 306), and the catheter (e.g., the intravascular reperfusion therapy device 160) has been removed from the patient's vasculature. In some cases, the stent 620 may be implanted as illustrated, and the configuration of the valve 621 may not be controlled via the processing system 110. For instance, the configuration of the valve 621 may remain fixed (e.g., at one of the first configuration 622 or the second configuration 624). In some embodiments, the configuration of the valve 621 may be adjusted by the flow of blood through the blood vessel. In some embodiments, the configuration of the valve 621 may be controlled remotely (e.g., wirelessly) by the processing system 110. For instance, as illustrated, the stent may include a sensor 710, a valve actuator 712, and a communication interface 714, which may be in communication with the processing system 110.

The sensor 710 may sense physiological data associated with blood flow through a blood vessel (e.g., the coronary vein 304). Further, sensor 710 may be substantially similar to the sensor 630. Accordingly, for the sake of brevity, details of the sensor 710 will not be repeated.

The valve actuator 712 may be a mechanical actuator or an electromechanical actuator coupled to the valve 621. In that regard, the valve actuator 712 may be arranged to apply a mechanical force to the valve 621 to control the configuration of the valve 621, and/or the valve actuator 712 may be arranged to supply an electrical signal to the valve 621 to control the configuration of the valve 621. In some embodiments, the valve actuator 712 may control the configuration of the valve 621 based on instructions from the processing system 110, which may be determined based on the physiological data obtained by the sensor 710, for example. Additionally or alternatively, the stent 620 may include a processor circuit 200 (e.g., a processor), such as a microprocessor, that is configured to instruct the valve actuator 712 to control the configuration of the valve 621. For instance the processor circuit 200 of the stent 620 may receive the physiological data obtained by the sensor 710 and instruct the valve actuator 712 based on this data. Accordingly, while techniques described herein refer to the processing system 110 receiving data and controlling aspects of the stent 620, embodiments are not limited thereto. In that regard, data may be received and/or obtained at the stent 620 itself, and the stent 620 may control the configuration of the valve 621 (e.g., via the processor circuit 200).

The communication interface 714 may facilitate communication between components of the stent 620 (e.g., the sensor 710 and/or the valve actuator 712) and the processing system. To that end, the communication interface 714 may be communicatively coupled to a communication module 214 of the processing system 110. Moreover, the communication interface 714 can include any electronic circuitry and/or logic circuitry to facilitate communication of data between various components of the stent 620 with one another and/or with the processing system 110. For instance, the communication interface 714 may include one or more antennas, electrical wires, transceivers, and/or the like. In some embodiments, the communication interface 714 can be an input/output (I/O) device interface, which may facilitate communicative coupling between the processing system 110 and the stent 620 and/or between components of the stent 620. Moreover, the communication interface 714 may facilitate wireless and/or wired communication between various elements of the stent 620, and the communication interface 714 may facilitate wireless communication between the stent 620 and the processing system 110. The communication interface 714 may facilitate such communication using any suitable communication technology, such as a cable interface such as a USB, micro USB, Lightning, or FireWire interface, Bluetooth, Wi-Fi, RF data transmission, ZigBee, Li-Fi, or cellular data connections such as 2G/GSM, 3G/UMTS, 4G/LTE/WiMax, or 5G.

FIG. 7B is a diagrammatic side view of an intravascular reperfusion therapy device 160 including an exemplary stent 620 that includes a valve 621. FIG. 7B includes features similar to those shown and described with respect to FIG. 7A. In the embodiment of the FIG. 7B, the intravascular reperfusion therapy device 160 includes a catheter or flexible elongate member 634 which is tethered, connected, and/or otherwise coupled to the stent 620. In that regard, the stent 620 and the catheter 634 can be permanently coupled such that, e.g., the stent 620 is not separable from the catheter 634. For example, the snare 633 can be support and/or interconnection structure that is permanently coupled to the stent body 628 and the catheter 634. In such embodiments, the stent 620 can lack the retrieval hook 632 (FIG. 7A). The stent 620 remains coupled to the catheter 634 during operation of the intravascular reperfusion therapy device 160 (e.g., opening and/or closing the valve 621).

The intravascular reperfusion therapy device 160 can include one or multiple communication lines 716. For example, the communication lines 716 can include conductive wires, conductors, or filars (carrying electrical signals), optical fibers (carrying optical signals), etc. The communication lines 716 can include insulating material and/or shielding material around the signal-carrying component (wire/conductor, optical fiber). In some embodiments, the communication lines 716 include a biocompatible material (e.g., a biocompatible polymer) around the communication lines 716, to allow the communication lines 716 to be exposed to tissue, blood, etc., inside the body lumen, such as a blood vessel. At a distal portion, the communication lines 716 can be mechanically and/or communicatively (e.g., electrically, optically) coupled to the sensor 710, the valve actuator 712, and/or the communication interface 714, respectively. At a proximal portion, the communication lines 716 can be mechanically and/or communicatively (e.g., electrically, optically) coupled to the processing system 110. The communication lines 716 can extend along a length of the catheter 634 from the proximal end of the catheter 634 to the distal end of the catheter 634. The communication lines 716 provide data and/or signal communication associated with the processing system 110, the stent 620, the valve 621, the sensor 710, the valve actuator 712, and/or the communication interface 714. For example, physiological data obtained by the sensor 710 can be transmitted to the processing system 101 via one or more communication lines 716. A command signal from the processing system 101 to the sensor 710 to activate the sensor 710 to obtain the physiological data can be transmitted via one or more communications lines 716. A command signal from the processing system to the valve actuator 712 to open or close the valve 621 (e.g., completely or to a particular degree) can be transmitted via one or more communications line 716. For example, the command signal may be an electrical signal (e.g., voltage and/or current) to control the state of electroactive polymer of the valve 621. An open status, closed status, degree of open/closed status of the valve 621 can be transmitted from the actuator 712 and/or the communication interface 714 to the processing system 101 via the one or more communication lines.

The communication lines 716 can be coupled to the stent 620 in a manner that does not interference with opening and/or closing of the valve 621. For example, the communication lines 716 can extend along the snare 633, which is coupled to the distal end of the catheter 634. In some embodiments, the communication lines 716 can extend along an external surface of the valve 621. While one respective communication line 716 is provided for each of the sensor 710, the valve actuator 712, and/or the communication interface 714 in the illustrated embodiment, it is understood that different quantities of communication lines and/or different ways of interconnection with the components of the valve 621 are contemplated. For example, multiple communication lines 716 can be provided for a one or more components. In some embodiments, multiple communication lines 716 are directly in communication with the communication interface 714 and indirectly in communication with the sensor 710 and the valve actuator 712. The communication interface 714 can be directly in communication with the sensor 710 and the valve actuator 712.

In some embodiments, one or multiple of the communication lines 716 are mechanical components, such as a pullwire or tendon (e.g., that does not carry an electrical or optical signal). For example, one or multiple of such lines 716 can be mechanically coupled to the actuator 712 such that the line 716 and the actuator 712 mechanically open and/or close the valve 621. A user can actuate a control mechanism (e.g., switch, knob, etc.), at a proximal portion of the intravascular reperfusion therapy device 160, to pull (increase tension) and/or release (decrease tension) the line 716, which causes the valve 621 to open and/or close (e.g., via the actuator 712). In this manner, the line 716 physically changes to respond to user control to open and/or close the valve 621. The line 716 can be made of, e.g., metal or polymer. For example, the line 716 can be a braided metal or polymer wire that is made up of multiple strands.

While the present disclosure describes embodiments of the intravascular reperfusion therapy device including a valve and/or a valve stent, it is understood that the intravascular reperfusion therapy device can include any suitable structure that selectively restricts blood flow and/or generates back pressure. For example, the structure can be the valve and/or valve stent. In some embodiments, the structure can be a controllable pump and a lumen for fluid (e.g., saline, oxygen). The structure can be an obstruction in some embodiments. For example, the obstruction can be an expandable structure (e.g., expandable balloon, expandable basket, expandable multi-arm structure with or without material in between the arms) that can be controlled to be in one state (expanded balloon, expanded basket, or expanded arms restricting blood flow) and another state (contracted balloon, contracted basket, or contracted arms allowing blood flow).

FIG. 8 is a flow diagram of a method 800 of evaluating (e.g., assessing) and controlling (e.g., modifying) a progress of reperfusion of a tissue, according to aspects of the present disclosure. In some embodiments, the method 800 may be used to control a reperfusion therapy, which may be delivered via a device, such as the intravascular reperfusion therapy device 160 of FIG. 1. In particular, the method 800 may be used to control an intravascular reperfusion therapy device positioned within a blood vessel associated with an area of tissue that reperfusion therapy is targeting, such as the device 160 and/or the stent 620 illustrated in FIG. 5. As an illustrative example, the blood vessel may be a coronary blood vessel, such as a coronary vein, and the area of tissue may be a myocardium (e.g., muscular tissue of the heart). The method 800 may additionally or alternatively be used to control a reperfusion therapy targeting microvascular disease (e.g., affecting capillary beds, for example) in any portion of a patient's anatomy (e.g., within or separate from the heart) and/or nonobstructive coronary artery disease. Further, in some cases, the area of tissue may be an area of tissue that receives an increase in blood flow, which may result from administration of a therapy, such as a PCI therapy.

As illustrated, the method 800 includes a number of enumerated steps, but embodiments of the method 800 may include additional steps before, after, or in between the enumerated steps. In some embodiments, one or more of the enumerated steps may be omitted, performed in a different order, or performed concurrently. The steps of the method 800 can be carried out by any suitable component within the system 100 and all steps need not be carried out by the same component. In some embodiments, one or more steps of the method 800 can be performed by, or at the direction of, a processor circuit of the system 100, including, the processing system 110 (e.g., the processor 210 (FIG. 2)) or any other component. For the purposes of example, method 800 is described with respect to features illustrated in FIGS. 1, 2, 4, 5, 6A-B, and 7. The description with respect to these features is intended to be illustrative and not limiting.

At step 802, the method 800 involves receiving physiological data associated with blood flow through a blood vessel. In particular, the physiological data may be associated with blood flow through a blood vessel that is associated with an area of tissue targeted for reperfusion therapy. As an illustrative example, the blood vessel may be a coronary blood vessel, such as a coronary vein (e.g., the coronary sinus 306 or another suitable vein), and the area of tissue may be a myocardium (e.g., muscular tissue of the heart) associated with the coronary vein. For instance, deoxygenated blood may flow from the myocardium to the associated coronary vein, as illustrated by the relationship of the area of tissue 310 with the coronary vein 304 and the coronary sinus 306 shown in FIGS. 4-5. For the purposes of example, the method 800 is described herein with respect to a myocardium (e.g., the area of tissue 310) and a coronary vein, such as the coronary sinus 306. However, embodiments are not limited thereto.

The processing system 110 may receive the physiological data from the one or more sensors (e.g., the sensor 630 and/or an additional sensor) of the intravascular reperfusion therapy device. Moreover, the one or more sensors may obtain the physiological data from within the blood vessel (e.g., intravascularly), as illustrated by the arrangement of the intravascular reperfusion therapy device 160 shown in FIG. 5. In particular, the received physiological data may be obtained from one or more sensors positioned on a flexible elongate member, such as catheter 634, and/or on the stent 620, such as sensor 710 (FIGS. 7A and 7B). Moreover, the processing system 110 may receive the physiological data while the one or more sensors are positioned within the blood vessel.

In some embodiments, the physiological data may include flow data, such as a flow rate or flow velocity of blood through the blood vessel. Additionally or alternatively, the physiological data may include pressure data, such as measurements of blood pressure within the blood vessel. Further, the physiological data may include data associated with the anatomy of the blood vessel, such as a diameter of the blood vessel, a distance between a component of the intravascular reperfusion therapy device and a wall of the blood vessel, and/or the like. In some embodiments, the physiological data may additionally or alternatively include intravascular imaging data, such as IVUS imaging data, OCT imaging data, and/or the like, obtained by the intravascular reperfusion therapy device 160 and/or the stent 620.

In some embodiments, the processing system 110 may further receive external imaging data associated with the blood vessel and/or the area of tissue associated with the blood vessel. For instance, the processing system 110 may receive imaging data obtained by the external imaging device 140, such as one or more include x-ray images, CT images, MRI images, SPECT images, external ultrasound images, and/or the like. The external imaging data may include contrast (e.g., may be obtained using contrast), which may be delivered by the contrast infusion pump 170, for example, such that blood flow and/or anatomical features may be visibly identified. In that regard, physiological data associated with the blood flow through the blood vessel may further be received and/or determined via external imaging data.

At step 804, the method 800 may involve determining a progression of a reperfusion therapy. That is, for example, a progression of a reperfusion therapy targeting an area of tissue (e.g., myocardium) associated with the blood vessel corresponding to the physiological data may be determined. In that regard, the reperfusion therapy may be a therapy delivered by the intravascular reperfusion therapy device 160 (e.g., via the stent 620) to an area of tissue 310 (e.g., a portion of myocardium). Moreover, the reperfusion therapy may regulate blood flow to the targeted tissue in some manner. In particular and as illustrated in FIG. 5, the intravascular reperfusion therapy device 160 (e.g., the stent 620) may generate a back pressure and/or back flow in a direction (e.g., indicated by arrow 626) opposite the flow of blood (e.g., indicated by arrow 504) within the blood vessel. The back pressure and/or back flow may change a pressure and/or resistance/impedance at the area of tissue 310, which may affect the distribution of blood flowing into (e.g., indicated by the arrow 502) the microvasculature (e.g., capillary bed) of the area of tissue 310. In that regard, the reperfusion therapy may be delivered such that blood suitably perfuses throughout the area of tissue 310, including to portions of the tissue 310 that are inflamed or damaged.

In some embodiments, determining a progression of the reperfusion therapy (e.g., at step 804) may involve determining a characteristic of blood flow through the blood vessel (e.g., a coronary vein 304) and/or through the area of tissue 310 (e.g., a portion of myocardium). In that regard, the processing system 110 may determine the progression of the reperfusion therapy delivered via the intravascular reperfusion therapy device 160 based on physiological data, such as physiological data received from the intravascular reperfusion therapy device 160 and/or external imaging data received from the external imaging device 140. For instance, based on the physiological data (e.g., pressure data, flow data, external imaging data, and/or the like), the processing system 110 may determine a flow velocity, a flow rate, and/or a pressure of blood within the blood vessel. The processing system 110 may additionally or alternatively determine a volume of blood and/or a resistance/impedance to blood flow within the blood vessel using the physiological data. For instance, the processing system 110 may determine the resistance and/or the impedance based on flow data and pressure data, in some embodiments.

The processing system 110 may relate a determined value (e.g., level) and/or change thereof of flow velocity, flow rate, pressure, volume, resistance/impedance, and/or the like to a progression of the reperfusion therapy. In general, the processing system 110 may associate a progression of a reperfusion therapy that produces values of such parameters or changes in these values indicating greater and/or increasing blood flow through the blood vessel as relatively effective (e.g., as having a positive and/or intended effect). Further, the processing system 110 may associate a progression of a reperfusion therapy that produces values of such parameters or changes in these values indicating lower, unchanging, and/or decreasing blood flow through the blood vessel as relatively ineffective (e.g., as having a negative and/or unintended effect).

In some embodiments, the processing system 110 may compare the determined values of such parameters (e.g., parameters associated with blood flow) to one or more thresholds or other values. As an illustrative example, a first range of values may correspond to healthy blood flow conditions, while a second range of values may correspond to unhealthy blood flow conditions. To that end, responsive to determining that a value of a parameter falls within the first range (e.g., satisfies a threshold), the processing system 110 may determine that the reperfusion therapy is complete, and responsive to determining that a value of a parameter falls within the second range (e.g., fails to satisfy a threshold), the processing system 110 may determine that additional reperfusion therapy may be delivered to the target area of tissue. In some embodiments, the first range of values and/or the second range of values may be determined on historical data associated with other patients, historical data associated with the patient, data associated with a healthy blood vessel (e.g., a vessel relatively unaffected by or not associated with the blockage 308) of the patient, and/or the like.

Additionally or alternatively, the processing system 110 may determine the change in value of such parameters over the delivery of the reperfusion therapy (e.g., over time) and may relate this change to the progression of the reperfusion therapy. In some embodiments, for example, the processing system 110 may compare a current value of a parameter with a set of one or more previous values of the parameter and/or determine a derivative of the parameter with respect to time. For instance, the processing system 110 may determine a derivative of the physiological data and/or of a parameter determined based on the physiological data with respect to time. To that end, the processing system 110 may determine whether a value of the physiological data and/or the parameter is increasing, decreasing, or relatively unchanging. Moreover, the processing system 110 may determine a rate of change of the value of the physiological data and/or the parameter. In this way, the processing system 110 may determine whether the reperfusion therapy is improving blood flow to the area of tissue 310 and/or the rate at which the reperfusion therapy is impacting the blood flow to the area of tissue 310. That is, for example, the processing system 110 may determine an efficacy and/or an efficiency of the reperfusion therapy.

In some embodiments, the processing system 110 may compare the change in the value of the parameter and/or the derivative of the parameter to one or more thresholds to determine a progression of the reperfusion therapy. For instance, a first range of values may correspond to the reperfusion therapy demonstrating improvement for (e.g., a positive effect on) the area of tissue, a second range may correspond to the reperfusion therapy demonstrating worsening blood flow conditions at the area of tissue, and a third range may correspond to the reperfusion therapy demonstrating having no effect or a relatively small effect on the area of tissue. Such thresholds may additionally or alternatively be used to quantify a degree of an effect the reperfusion therapy has on the tissue area. Moreover, the processing system 110 may use the physiological data and/or derivatives thereof to determine when the reperfusion therapy is complete by determining whether the value of one or more parameters has reached a healthy threshold or has plateaued at a maximum value.

Further, the progression of the reperfusion therapy may additionally or alternatively be determined based on external imaging data obtained by the external imaging device 140. For instance, in some embodiments, the processing system 110 may determine changes in blood flow (e.g., changes in flow velocity, flow rate, and/or the like) through a vessel over time (e.g., before, during, and/or after delivery of reperfusion therapy) based on external imaging data (e.g., from the external imaging device 140). For instance, using imaging processing techniques, the processing system 110 may identify a change in a blood flow through a blood vessel based on a change in an intensity, contrast, brightness, color, and/or the like of an area (e.g., an image element and/or a pixel) of external image data obtained with contrast. An example of image processing includes conducting a pixel level analysis to evaluate whether there is a change in the color of the pixel between different images in external image data captured over different time points. Continuing with this example, for external imaging data obtained with a contrast that decreases exposure of blood (e.g., on an x-ray detector in conjunction with the x-ray source), a change in pixel color from a lighter color (e.g., gray or white) to a darker color (e.g., black) within different external images may indicate an increase in blood flow (e.g., blood perfusion) through an area (e.g., blood vessel and/or tissue) associated with the pixel, which may correspond to a progression of the reperfusion therapy providing improvement to a patient's health. A change in pixel color from a darker color to a lighter color, on the other hand, may indicate a decrease in blood flow through the area, which may correspond to a progression of the reperfusion therapy providing a negative effect on a patient's health. Moreover, as similarly described with the values of physiological data and/or parameters associated with the data, changes in blood flow and/or external imaging data characteristics (e.g., intensity, contrast, brightness, and/or the like) identified based on external imaging data may be compared to one or more thresholds and/or may be further evaluated to determine the progression of the reperfusion therapy and/or a rate of the progression of the reperfusion therapy.

At step 806, the method 800 may involve controlling the reperfusion therapy based on the determined progression of the reperfusion therapy (e.g., based on the physiological data associated with the blood flow through the blood vessel). In particular, at step 806, a configuration of the valve 621 and/or a configuration (e.g., an expansion state) of the stent 620 may be controlled to control the reperfusion therapy. In some embodiments, for example, the processing system 110 may control and/or instruct the intravascular reperfusion therapy device 160 based on the progression of the reperfusion therapy. For instance, the processing system 110 may control and/or instruct the intravascular reperfusion therapy device to adjust the configuration of the valve 621 may from the first configuration 622 to the second configuration or vice versa and/or to deploy or retrieve the stent 620 (e.g., to adjust the stent 620 between a collapsed or expanded expansion state). In this regard, to control the configuration of the valve 621 and/or the expansion state of the stent 620, the processing system 110 may control an electrical signal, thermal energy, and/or a mechanical force supplied to the intravascular reperfusion therapy device 160 and/or the stent 620.

In some embodiments, based on the determined progression of the reperfusion therapy indicating that the progression is relatively poor, not effective, and/or that blood flow is not improving in the blood vessel, the processing system 110 may control the stent 620 (e.g., the intravascular reperfusion therapy device 160) to increase back pressure resulting on the venous side of the tissue (e.g., increase venous obstruction). In some embodiments, to increase the back pressure, the processing system 110 may instruct the intravascular therapy device 160 to deploy the stent 620, which may transition the stent from a collapsed state to an expanded state. In the deployed, expanded state, the stent 620 may obstruct blood flow, increasing the back pressure. Further, the processing system 110 may instruct the intravascular reperfusion therapy device 160 to adjust and/or control the configuration of the valve 621. For instance, the processing system 110 may instruct the intravascular reperfusion therapy device 160 to adjust the configuration of the valve 621 such that a degree of obstruction provided by the valve 621 within a blood vessel is increased. As an illustrative example, the processing system 110 may instruct the intravascular reperfusion therapy device 160 to transition the valve 621 from the first configuration 622 to the second configuration 624 to increase the back pressure and adjust the reperfusion therapy. Responsive to such instruction, the intravascular reperfusion therapy device 160 may provide an electrical signal, apply thermal energy, apply a mechanical force, and/or the like to control the configuration of the valve 621, as described above. Further, in some embodiments, the processing system 110 may control the intravascular reperfusion therapy device 160 to alter or initiate an oscillation of the valve 621 between different configurations to adjust the delivery of the reperfusion therapy. For instance, in such embodiments, the processing system 110 may instruct the intravascular reperfusion therapy device 160 to increase a period of the duty cycle and/or a frequency that the valve 621 is in the second configuration 624 with respect to the first configuration 622, which increase the back pressure and/or back flow, as well as an aggressiveness of the reperfusion therapy.

Moreover, in some embodiments, the expansion state of the stent 620 may be oscillated. In that regard, the intravascular reperfusion therapy device 620 may periodically (e.g., regularly) or aperiodically deploy and retract (e.g., retrieve) the stent 620. To that end, to increase the back pressure, the processing system 110 may instruct the intravascular reperfusion therapy device 160 to increase a period of the duty cycle and/or a frequency that the stent 620 is in the expanded state. In this way, the period and/or frequency at which the stent 620 obstructs the blood flow of a blood vessel (e.g., via the valve 621) may be increased. In some embodiments, the processing system 110 may control the expansion state of the stent 620 alone or in combination with control of the configuration of the valve 621. For instance, in some embodiments, the expansion state of the stent 620 may be controlled, while the configuration of the valve 621 may remained fixed. In some embodiments, when the stent 620 is oscillated between a collapsed and expanded state, the configuration of the valve 621 may be oscillated while the valve 621 is arranged in the expanded state.

Based on the determined progression of the reperfusion therapy indicating that the progression is relatively good, effective, and/or that the blood flow is improving in the blood vessel, the processing system 110 may instruct the stent 620 to maintain a current therapy (e.g., oscillation pattern and/or degree of expansion) and/or to gradually terminate (e.g., ease off) delivery of the therapy. Continuing with the above example, decreasing the period of the duty cycle and/or frequency that the stent 620 is expanded and/or that the valve 621 is arranged in the second configuration 624 may decrease the back pressure and/or back flow, as well as an aggressiveness of the reperfusion therapy. Additionally or alternatively, the processing system 110 may control the valve 621 to transition to a configuration (e.g., the first configuration 622) with a greater diameter available for blood flow responsive to determining that the reperfusion therapy is relatively good, effective, and/or that blood flow through the vessel is improving.

In general, the processing system 110 may adjust one or more characteristics of the intravascular reperfusion therapy device 160 operation (e.g., configuration of the stent 620) based on a feedback loop with the intravascular reperfusion therapy device 160 and the determined progression of the reperfusion therapy. In that regard, it may be appreciated that steps of the method 800 may be repeated, such that the processing system 110 may continually determine a current progression of the reperfusion therapy (e.g., based on updated physiological data) and adaptively adjust the operation of the intravascular reperfusion therapy device 160 and/or the configuration of the stent 620.

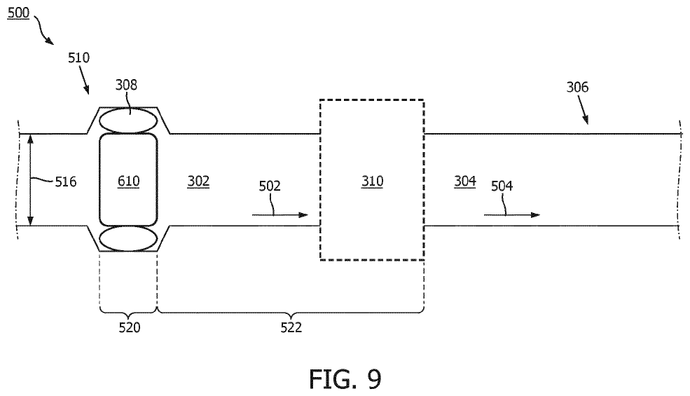

In some embodiments, responsive to determining that the blood flow through the first area has reached a healthy level (e.g., satisfies a threshold), has reached a level substantially similar to blood flow through the second area, has plateaued at a maximum value, the processing system 110 may determine that the progression of the reperfusion therapy has reached completion. In such cases, the processing system 110 may control the intravascular reperfusion therapy device 160 to terminate delivery of the reperfusion therapy. For instance, the processing system 110 may instruct the intravascular reperfusion therapy device 160 and/or provide a recommendation (e.g., via the display 120) to retrieve the stent 620 to terminate the delivery of the reperfusion therapy (e.g., responsive to determining the therapy has reached completion). FIG. 9 provides an illustrative example of the portion 500 of the heart after reperfusion therapy is complete, as illustrated by the absence of the intravascular reperfusion therapy device 160. In that regard, FIG. 9 may correspond to a detailed view of a portion of the heart 300 illustrated in FIG. 3C. As described above, when reperfusion therapy is complete, the blood flow in the first area of tissue 310 may be greater than a blood flow amount following a PCI therapy and/or may be substantially similar to the blood flow in healthy tissue, such as tissue unaffected and/or not associated with a vessel including a blockage.

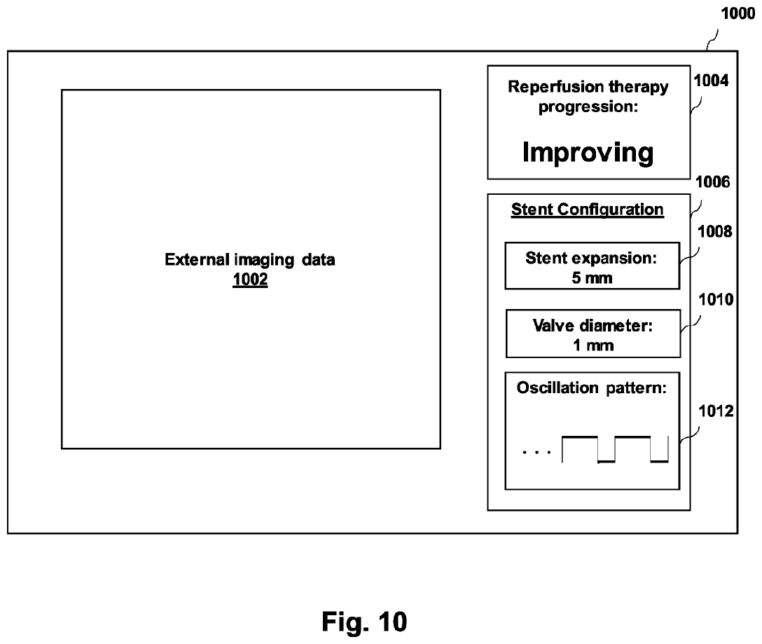

With reference again to FIG. 8, at step 808, the method 800 may optionally involve (as indicated by the dashed lines) outputting a visual representation of the progression of the reperfusion therapy to a display. For instance, the processing system 110 may output a screen display (e.g., a graphical user interface (GUI)) including a representation of the progression of the reperfusion therapy to the display device 120. An example of a screen display including a representation of the progression of the reperfusion therapy is illustrated and described with respect to FIG. 10.

FIG. 10 illustrates a screen display 1000, which includes a graphical representation of external imaging data 1002, a visual representation of the progress of the reperfusion therapy 1004, and a visual representation of the configuration of the stent 1006 (e.g., the expansion state of the stent 620 and/or the configuration of the valve 621). The external imaging data 1002 may be obtained by the external imaging device 140, as described above, and may be output to the display 120 by the processing system 110. The external imaging data 1002 may depict the area of tissue 310, a coronary vein 304 associated with the area of tissue 310 (e.g., the coronary sinus 306), and/or a coronary artery 302 associated with the area of tissue 310. Moreover, the external imaging data 1002 may include contrast in some embodiments. In some embodiments, the delivery and/or progress of a reperfusion therapy may be visualized via the external imaging data 1002.

In some embodiments, the visual representation of the progression of the reperfusion therapy 1004 may include a visual representation of the physiological data and/or a value of a parameter determined based on the physiological data. In that regard, the visual representation of the progression of the reperfusion therapy 1004 may include a representation of blood flow through the blood vessel. In some embodiments, the visual representation of the progression of the reperfusion therapy 1004 may include a numerical representation, a graph, chart, or plot, a textual representation, one or more symbols, and/or the like. As further illustrated, in some embodiments, the determined progression of the reperfusion therapy (e.g., at step 804) may be represented textually as a particular status, such as “improving,” “worsening,” “no change,” “complete,” and/or the like. In some embodiments, each status may correspond to a different threshold or range of values of the physiological data, parameters determined based on the data, derivations, external imaging data and/or the like. For instance, as described above, the status “improving” may correspond to a first range of values for physiological data and/or an associated parameter that correspond to improvement for the area of tissue 310, the status “worsening” may correspond to a second range of values for the physiological data and/or associated parameter that correspond to worsening blood flow conditions associated with the area of tissue 310, the status “no change” may correspond to a third range of values for the physiological data and/or the associated parameter that correspond to the reperfusion therapy demonstrating no effect or a relatively small effect on the area of tissue 310, and the status “complete” may correspond to a fourth range of values for the physiological data and/or the associated parameter that correspond to a healthy level of blood flow associated with the area of tissue 310 and/or to a plateaued maximum value. Further, the progression of the reperfusion therapy may additionally or alternatively be indicated with respective symbols, colors, and/or the like associated with the statuses “improving,” “worsening,” “no change,” “complete,” and/or the like. Moreover, the screen display 1000 may include external imaging data 1002, such as one or more static images or an image stream. In some embodiments, navigating through the static images (e.g., using the input device 130) or watching a progression of the image stream may provide an indication of the progress of the reperfusion therapy, as the visual appearance of contrast delivered to the area of tissue will change based on an impact of the reperfusion therapy.

The visual representation of the configuration of the stent 1006 may display a visual representation of a degree of expansion of the stent 1008 (e.g., a diameter of the stent 620), a visual representation of a configuration of the valve 1010 (e.g., a diameter of the valve 621), a visual representation of an oscillation pattern 1012 of the valve 621 between different configurations and/or the stent 620 between expansion states, and/or the like. In some embodiments, visual representation of a degree of expansion of the stent 1008 may be a visual representation of a set diameter, such as a diameter the processing system 110 controls the stent 620 to expand to, a diameter a user selects (e.g., via a user input at the input device 130) the stent 620 to expand to, and/or the like. To that end, the processing system 110 may determine the visual representation of a degree of expansion of the stent 1008 based on the set diameter determined by the processing system 110 for control of the stent 620 in therapy delivery (e.g., at step 806 of the method 800) and/or a diameter selected by a user. The visual representation of a degree of expansion of the stent 1008 may additionally or alternatively be a visual representation of an actual diameter of the stent 620, which may be sensed by the intravascular reperfusion therapy device 160 (e.g., via the sensor 630, for example), the stent 620 (e.g., via the sensor 710), and/or determined based on the external imaging data 1002. In some embodiments, for example, at least a portion of the stent 620 may be radiopaque. Accordingly, the stent 620 may be visualized via external imaging data 1002, and the processing system 110, using the external imaging data 1002 and image processing techniques (e.g., pixel level image processing), may determine the actual diameter of the stent 620. Thus, the illustrated visual representation of a degree of expansion of the stent 1008 may represent the set or the actual diameter of the stent 620. Moreover, while a single diameter is illustrated, embodiments are not limited thereto. In that regard, a visual representation of the actual diameter and the set diameter may be output to the display 120 in some embodiments. Similarly, the visual representation of the configuration of the valve 1010 may be a visual representation of an actual diameter of the valve 621, which may be determined based on image processing of the external imaging data 1002, sensed by the sensor 630, sensed by the sensor 710, and/or the like, a visual representation of a set diameter of the valve 621, or both. As further illustrated, the visual representation of the oscillation pattern 1012 may include a waveform of one or more of the oscillation patterns. Additionally or alternatively visual representation of the configuration of the stent 1006 may include a numerical representation, a graph, chart, or plot, a textual representation, one or more symbols, and/or the like. In some embodiments, the passage or obstruction of blood flow caused by the stent 620 may additionally or alternatively be visualized via the external imaging data 1002 (e.g., obtained with contrast).

Based on the visual representation output to the screen display, a user, such as a physician or clinician, may monitor the progression of the reperfusion therapy. Moreover, in some embodiments, the user may also control or modify the reperfusion therapy. For instance, the reperfusion therapy may be further controlled based on one or more user inputs, which may be received via the input device 130, for example. To that end, a user may provide an input at the input device 130, and the processing system 110 may control operation of the intravascular reperfusion therapy device 160 based on the input.

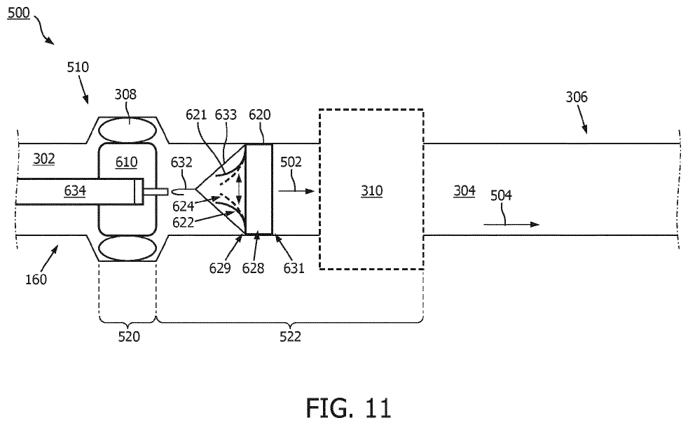

Turning to FIG. 11, a schematic diagram of the portion 500 of the heart during and/or following delivery of a PCI therapy 610 and/or during delivery of a reperfusion therapy is shown. In that regard, FIG. 11, like FIG. 5, may correspond to a detailed view of a portion of the heart 300 illustrated in FIG. 3B. However, while the reperfusion therapy is illustrated in FIG. 5 as being supplied within the venous vasculature (e.g., the coronary sinus 306), the delivery of reperfusion therapy illustrated in FIG. 11 is supplied via the arterial vasculature, such as within the coronary artery 302. That is, for example, the stent 620 is illustrated as being positioned within the coronary artery 302. In that regard, while the method 800 of FIG. 8 is described with respect to venous delivery of reperfusion therapy, the method 800 may additionally or alternatively be employed with respect to arterial delivery of reperfusion therapy, as described below.

As illustrated in FIG. 11, to deliver reperfusion therapy arterially (e.g., on the arterial side of the area of tissue 310), the intravascular reperfusion therapy device 160 and/or the stent 620 may be positioned within the coronary artery 302. Because characteristics of the blood flow through the coronary artery 302 may vary in comparison with the blood flow through the coronary veins 304, the physiological data and/or parameters associated with blood flow derived from the physiological data may be compared against different thresholds or otherwise related to a progression of a reperfusion therapy (e.g., at step 804 of the method 800) differently than the corresponding data during venous reperfusion therapy. In general, reperfusion therapy targeting the area of tissue 310 may increase blood flow through the area of tissue 310 by, for example, reducing resistance and/or impedance to flow within the area of tissue 310. Accordingly, physiological data demonstrating that blood flow through the area of tissue 310 is increasing may indicate a progression of a reperfusion therapy that is effective and/or achieving an intended effect, while physiological data demonstrating that blood flow through the area of tissue 310 is decreasing or unchanging may indicate a progression of a reperfusion therapy that is effective and/or achieving an intended effect.

The intravascular reperfusion therapy device 160 may be the same as or different from the intravascular lesion therapy device 150. In that regard, the intravascular reperfusion therapy device 160 may be configured to deliver the reperfusion therapy after a PCI therapy 610 is delivered and/or the intravascular reperfusion therapy device 160 may deliver both the PCI therapy 610 as well as reperfusion therapy. As an illustrative example, the intravascular reperfusion therapy device 160 may deploy a stent 620 with a valve 621 to both deliver the PCI therapy 610 and the reperfusion therapy. For instance, the stent 620 may be positioned at the portion 520 of the vessel with the blockage 308, and expansion of the stent 620 to the expanded state may increase the diameter of the portion 520 of the vessel (e.g., to the diameter 516). While the stent 620 may increase the diameter of the portion 520 of the vessel, the valve 621 may restrict blood flow to pass through a dimension (e.g., a diameter) of the valve, which may be less than the diameter of the portion 520. Controlling the configuration of the valve 621 may thus control the dimension (e.g., diameter) blood flow is able to pass through. In that regard, by modifying the valve 621 configuration to increase the dimension for blood flow, such as modifying the valve 621 from the second configuration 624 to the first configuration 622, the blood flow to the area of tissue 310 may be increased. Moreover, by controlling the rate of change between different valve configurations, the rate of blood flow changes may be controlled. In this manner, blood flow to the area of tissue 310 may be restored in a controlled and/or gradual manner after delivery of a PCI therapy 610 (e.g., the stent 620), which may prevent or reduce reperfusion injury. Moreover, in some embodiments, after the reperfusion therapy is delivered, the valve 621 may be set to a fully open configuration, where the diameter of the valve 621 permitting blood flow may substantially match the diameter of the stent body 628. In this way, the stent 620 may be relatively permanently implanted in a patient to continue delivering the PCI therapy 610 after the reperfusion therapy is completed. Thus, in some embodiments, delivery of the reperfusion therapy (e.g., the stent 620) may be co-located with the blockage 308 and/or the site where the PCI therapy 610 is delivered. In some embodiments, the stent 620 may be positioned between the portion 520 of the vessel where the blockage 308 and/or PCI therapy 610 is located and the area of tissue 310, as illustrated. That is, for example, the stent 620 may be positioned distal of the blockage 308 and/or the PCI therapy 610 (e.g., within the distal area 522). Additionally or alternatively, the stent 620 may be positioned proximal of the portion 520 of the vessel where the blockage 308 and/or PCI therapy 610 is located.

In cases where the reperfusion therapy is delivered arterially, the intravascular reperfusion therapy device 160 may be configured to obstruct arterial vasculature (e.g., the coronary artery 302) at varying degrees to control the flow of blood into the area of tissue 310. As an illustrative example, the intravascular reperfusion therapy device 160 may employ the stent 620 to obstruct the coronary artery 302 to a first degree via the valve 621, as indicated by the second configuration 624, and as the valve 621 is transitioned to the first configuration 622 (e.g., as the diameter of the stent valve 621 is increased), increased blood flow may be provided to the area of tissue 310. On the other hand, transitioning the valve 621 to a configuration with a smaller diameter may decrease blood flow to the area of tissue. In this way, blood flow to the tissue 310 may be gradually increased or decreased via control of the stent 620 (e.g., by the processing system 110). To that end, based on the progression of the reperfusion therapy, the processing system 110 may control the reperfusion therapy by adjusting a degree of obstruction (e.g., expansion) within the vasculature caused by the stent 620 (e.g., via the valve) and/or by controlling a rate of change between configurations of the valve 621 (e.g., at step 806 of the method 800). In some embodiments, for example, gradually increasing blood flow through the tissue 310 may minimize or prevent reperfusion injury from occurring and/or may reverse an effect of reperfusion injury. In that regard, in cases where the intravascular reperfusion therapy device 160 is used to deliver the PCI therapy 610, the stent 620 may be expanded during delivery of the PCI therapy 610, and subsequently, the processing system 110 may control the valve 621 to gradually reintroduce blood flow to the tissue 310 (e.g., to gradually widen the diameter of the valve 621 that permits blood flow). Further, as described above, the processing system 110 may vary the expansion state of the stent 620 and/or the rate of change between expansion states of the stent 620 to control the intravascular reperfusion therapy. Moreover, the processing system 110 may control the delivery of reperfusion therapy based on a feedback loop with the intravascular reperfusion therapy device 160 and/or the stent 620 (e.g., based on the sensed physiological data). In addition, mechanisms to adjust reperfusion therapies described herein are intended to be illustrative and not limiting.

FIGS. 12A-12B illustrate exemplary arrangements of the one or more sensors (e.g., the sensor 630) of the intravascular reperfusion therapy device 160. In particular, FIGS. 12A-12B illustrate a front view of a cross section of a catheter 634 of the intravascular reperfusion therapy device 160. In an exemplary embodiment, FIG. 12A illustrates a front view of the sensor 630 positioned on a distal end 635 of the catheter 634, where the catheter 634 terminates. In other embodiments, the sensor 630 and the illustrated cross section of FIG. 12A may be spaced from the distal end of the catheter. Further, in an exemplary embodiment, FIG. 12B illustrates a front view of the sensor 630 and the second sensor 1102 positioned on a distal end of the catheter 634. In other embodiments, the sensor 630, the second sensor 1102, and the illustrated cross section may be spaced from the distal end of the catheter 634. Moreover, while FIGS. 12A-12B are illustrated and described with respect to positioning of sensors on a catheter, embodiments are not limited thereto. In that regard, the illustrated sensors may additionally or alternatively be arranged on a guidewire according to the techniques described herein. Additionally or alternatively, the arrangements of the sensors illustrated and described with respect to FIGS. 12A-12B may be applicable to the sensor 710 of the stent 620 illustrated in FIGS. 7A and 7B.

In some embodiments, the one or more sensors of the intravascular reperfusion therapy device 160 may include one or more transducers, such as one or more ultrasound transducer elements. To that end, the illustrated configurations of FIGS. 12A-12B may be representative of one or more transducers. The one or more ultrasound transducer elements (e.g., acoustic elements) may be configured to emit ultrasound energy and receive echoes corresponding to the emitted ultrasound energy. Further, the one or more ultrasound transducer elements may include a piezoelectric/piezoresistive element, a piezoelectric micromachined ultrasound transducer (PMUT) element, a capacitive micromachined ultrasound transducer (CMUT) element, and/or any other suitable type of ultrasound transducer element. The one or more ultrasound transducer elements may further be in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can include a microbeamformer (uBF). In other embodiments, one or more of the ICs includes a multiplexer circuit (MUX).

Further the one or more transducers of the one or more sensors may be arranged in any suitable configuration. For example, the transducers may be positioned in an array of ultrasound transducer elements, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

In an exemplary embodiment, the sensor 630 or a second sensor 1102 is a flow sensor, which includes a single ultrasound transducer element, such as the transducer elements described above. The transducer element emits ultrasound signals and receives ultrasound echoes reflected from anatomy (e.g., flowing fluid, such as blood). The transducer element generates electrical signals representative of the echoes. These electrical signals are carried from the sensor at the distal portion of the device 160 to a connector at the proximal portion of the device 160, which may be communicatively coupled to the processing system 110. The processing system 110 can then process the electrical signals to extract the flow velocity of the fluid.

In some embodiments, the sensor 630 or the second sensor 1102 is thermal anemometric sensor, such as a thermal anemometric flow sensor. In such cases, the sensor may be maintained at a relatively constant temperature, and the electrical power consumed to maintain this temperature as blood flows proximate the sensor may be sensed and as representative of the flow velocity of the blood flow. Alternatively, a relatively constant power may be supplied to the sensor, and a change in temperature of the sensor as the blood flows proximate the sensor may be sensed as representative of the flow velocity of the blood flow. For instance, the processing system 110 may relate the consumed power or the temperature change, respectively to determine the flow velocity of the blood. While the sensors are described as being implemented with transducer elements or as a thermal anemometric sensor, embodiments are not limited thereto. In that regard, any suitable sensor configured to obtain physiological data associated with blood flow may be utilized in the intravascular reperfusion therapy device 160.

As shown in FIG. 12A, the sensor 630 is arranged in an annular configuration about (e.g., following an outer perimeter of) the catheter 634. This annular configuration may include one or more transducers, such as one or more ultrasound transducers, as described above. Further, in some embodiments, the transducer elements and/or the sensor 630 may be forward-facing. That is, for example, the sensor 630 may be a flow sensor arranged so that the sensor 630 senses (e.g., emits signals) along the axis blood flows along within the vessel (e.g., the axis including the blood flow directions indicated by arrow 502 and arrow 504 in FIGS. 4-5 and 9-10), which may correspond to the longitudinal axis of the intravascular reperfusion therapy device). In that regard, the sensor 630 may be arranged to sense in a direction aligned with or opposite the direction of blood flow within the vessel. In some embodiments, the sensor 630 may be substantially perpendicular with the longitudinal axis of the intravascular reperfusion therapy device 160 to be forward-facing. In some embodiments, the sensor 630 may be positioned at an oblique angle (e.g., tilted) such that at least a portion of the signals emitted by the sensor 630 detect physiological data with respect to the axis that blood flows along within the vessel. For instance, in cases where the sensor 630 is spaced from the distal end of the intravascular reperfusion therapy device 160, the sensor 630 may be positioned at an angle to detect blood flow.

FIG. 12B, illustrates the sensor 630 and a second sensor 1102 positioned on the catheter 634. Again, the illustrated sensor 630 may represent one or more transducers, and the illustrated second sensor 1102 may represent one or more transducers. Moreover, the sensor 630 and the second sensor 1102 may be the same or different. For instance, the sensor 630 may be a flow sensor, as described above, and the second sensor 1102 may be a flow sensor or a pressure sensor.

In some embodiments, the sensor 630 and the second sensor 1102 may be spaced from one another along the circumference of the catheter 634, as illustrated. Further, the sensor 630 and the second sensor 1102 may face the same or different directions. For instance, the sensor 630 may be entirely forward facing (e.g., perpendicular to the longitudinal axis of the device 160), while the second sensor 1102 may be tilted or parallel with the axis of the device 160. The second sensor 1102 may be a pressure sensor arranged parallel with the axis of the device 160, for example.

Further, in some embodiments, the sensor 630 and the second sensor 1102 may be positioned within the catheter 634, such as within the lumen 690 of the catheter 634 and/or within a housing positioned within or defined by the lumen 690. In some embodiments, the sensor 630 and the second sensor 1102 may be embedded within a material of the catheter 634 or positioned on an outer surface of the catheter 634. In that regard, the sensor 630 and the second sensor 1102 may be arranged in any suitable configuration for collecting physiological data intravascularly.

While the system 100 and the method 800 are described herein as being employed for evaluating (e.g., assessing) and/or controlling reperfusion therapy, embodiments are not limited thereto. In that regard, the techniques described herein may additionally or alternatively be applied to microvascular disease (e.g., affecting capillary beds, for example) in any portion of a patient's anatomy (e.g., within or separate from the heart) and/or to nonobstructive coronary artery disease. Moreover, assessment and/or control of blood flow through particular tissue and/or capillary beds may be performed with or without a PCI therapy being performed on a vessel associated with the tissue and/or capillary beds.

A person of ordinary skill in the art will recognize that the present disclosure advantageously provides a system and method suitable to evaluate (e.g., assess) and control (e.g., adjust) a reperfusion therapy associated with an area of tissue. In particular, the techniques described herein enable adaptive delivery of a reperfusion therapy via an intravascular stent with a valve based on intravascularly sensed physiological data. Moreover, the stent with the valve may advantageously be implanted within a patient over a duration (e.g., one or more hours or days) suitable to deliver a reperfusion therapy. The logical operations making up the embodiments of the technology described herein are referred to variously as operations, steps, objects, elements, components, regions, etc. Furthermore, it should be understood that these may occur in any order, unless explicitly claimed otherwise or a specific order is inherently necessitated by the claim language.

It should further be understood that the described technology may be employed in a variety of different applications, including but not limited to human medicine, veterinary medicine, education and inspection. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal imaging system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a stent configured to be positioned within a coronary vein of a patient, comprising:
a valve; and
a stent body coupled to the valve and arranged to transition an expansion state of the stent between a collapsed state and an expanded state;
an intravascular reperfusion therapy device configured to be positioned within the coronary vein, wherein the intravascular reperfusion therapy device comprises a sensor and a catheter comprising a lumen shaped to receive the stent, wherein the intravascular reperfusion therapy device is configured to deliver, using the stent, reperfusion therapy to a myocardium of a heart of the patient associated with the coronary vein; and
a processor circuit in communication with the intravascular reperfusion therapy device and configured to:
receive, from the sensor, physiological data associated with blood flow through the coronary vein;
determine, based on the physiological data, a progression of the reperfusion therapy delivered to the myocardium of the heart; and
control, based on the progression of the reperfusion therapy, at least one of a configuration of the valve or the expansion state of the stent such that back pressure within the coronary vein is controlled,
wherein, with the stent in the expanded state:
the stent body is configured to contact a wall of the coronary vein; and
the valve is arranged to deliver the reperfusion therapy by obstructing blood flow in a first direction in the coronary vein to generate the back pressure in an opposite, second direction in the coronary vein.

2. The system of claim 1, wherein, with the stent in the expanded state, the valve transitions a diameter of the stent from a first diameter to a second diameter less than the first diameter.

3. The system of claim 2, wherein the valve comprises a windsock valve.

4. The system of claim 2, wherein the valve comprises a duckbill valve.

5. The system of claim 1, wherein the valve is coupled to a proximal end of the stent body.

6. The system of claim 1, wherein the stent further comprises a retrieval hook such that the stent is configured to be removably positioned within the coronary vein, wherein the retrieval hook is coupled to a proximal end of the stent body.

7. The system of claim 6, wherein the retrieval hook is arranged such that, responsive to a mechanical force on the retrieval hook with the stent in the expanded state, the stent body transitions the stent to the collapsed state.

8. The system of claim 1, wherein, in a first configuration, the valve is arranged to obstruct the blood flow in the first direction to a first degree, and, in a different, second configuration, the valve is arranged to obstruct the blood flow in the first direction to a different, second degree.

9. The system of claim 8, wherein the valve comprises an electroactive polymer configured to transition the valve from the first configuration to the second configuration in response to an electrical signal, and wherein, to control the configuration of the valve, the processor circuit is configured to control the electrical signal.

10. The system of claim 1, wherein the processor circuit is further configured to:
output, to display in communication with the processor circuit, a visual representation of the progression of the reperfusion therapy.

11. The system of claim 1, wherein the sensor comprises a flow sensor, wherein the physiological data comprises a blood flow rate.

12. The system of claim 1, wherein the sensor comprises a pressure sensor, wherein the physiological data comprises pressure data.

13. The system of claim 1, wherein the sensor comprises an ultrasound transducer positioned at a distal portion of the catheter.

14. The system of claim 1, wherein the stent body comprises at least one of a wire mesh or a self-expanding material.

15. The system of claim 1, wherein the coronary vein comprises a coronary sinus.

16. The system of claim 1, wherein, to determine the progression of the reperfusion therapy, the processor circuit is configured to:
determine a derivative of the physiological data with respect to time.

17. A system, comprising:
a stent configured to be positioned within a coronary vein of a patient, comprising:
a valve; and
a stent body coupled to the valve and arranged to transition an expansion state of the stent between a collapsed state and an expanded state;
an intravascular reperfusion therapy device configured to be positioned within the coronary vein, wherein the intravascular reperfusion therapy device comprises:
a flow sensor positioned at a distal portion of the intravascular reperfusion therapy device; and
a catheter comprising a lumen shaped to receive the stent,
wherein the intravascular reperfusion therapy device is configured to deliver, using the stent, reperfusion therapy to a myocardium of a heart of the patient associated with the coronary vein; and
a processor circuit in communication with the intravascular reperfusion therapy device and configured to:
receive, from the flow sensor, flow data associated with blood flow through the coronary vein;
determine, based on the flow data, a progression of the reperfusion therapy delivered to the myocardium of the heart; and
control, based on the progression of the reperfusion therapy, at least one of a configuration of the valve or the expansion state of the stent such that back pressure within the coronary vein is controlled,
wherein, with the stent in the expanded state:
the stent body is configured to contact a wall of the coronary vein; and
the valve is arranged to deliver the reperfusion therapy by obstructing blood flow in a first direction in the coronary vein to generate the back pressure in an opposite, second direction in the coronary vein,
wherein the valve is arranged to obstruct the blood flow in the first direction to a first degree in a first configuration of the configuration of the valve and the valve is arranged to obstruct the blood flow in the first direction to a different, second degree in a second configuration of the configuration of the valve.

18. A stent removably positionable within a coronary vein, comprising:
a valve;
a retrieval hook; and
a self-expanding material coupled to the valve and the retrieval hook and arranged to transition the stent between a collapsed state and an expanded state,
wherein, with the stent in the expanded state:
the self-expanding material is configured to contact a wall of the coronary vein; and
the valve is arranged to obstruct blood flow in a first direction in the coronary vein to generate back pressure in an opposite, second direction in the coronary vein.

19. An apparatus, comprising:
a stent configured to be positioned within a coronary vein of a patient, comprising:
a valve; and
a stent body coupled to the valve and arranged to transition an expansion state of the stent between a collapsed state and an expanded state;
an intravascular reperfusion therapy device configured to be positioned within the coronary vein, wherein the intravascular reperfusion therapy device comprises a catheter comprising a lumen shaped to receive the stent, wherein the intravascular reperfusion therapy device is configured to deliver, using the stent, reperfusion therapy to a myocardium of a heart of the patient associated with the coronary vein,
wherein, with the stent in the expanded state:
the stent body is configured to contact a wall of the coronary vein; and
the valve is arranged to deliver the reperfusion therapy by obstructing blood flow in a first direction in the coronary vein to generate the back pressure in an opposite, second direction in the coronary vein.

20. The apparatus of claim 19, wherein, in a first configuration, the valve is arranged to obstruct the blood flow in the first direction to a first degree, and, in a different, second configuration, the valve is arranged to obstruct the blood flow in the first direction to a different, second degree.

21. The apparatus of claim 20, wherein the valve comprises an electroactive polymer configured to transition the valve from the first configuration to the second configuration in response to an electrical signal.

22. The apparatus of claim 21, wherein the system further comprises a processor circuit configured to provide the electrical signal to control the configuration of the valve.
